(19) United States
(12) Patent Application Publication
(10) Pub. No.: US 2012/0241620 A1
(43) Pub. Date: Sep. 27, 2012

(54) OPTICAL CONTROL DEVICE, CONTROL DEVICE, AND OPTICAL SCOPE

(75) Inventor: Seigo ON, Tokyo (JP)

(73) Assignee: OLYMPUS CORPORATION, Tokyo (JP)

(21) Appl. No.: 13/486,182

(22) Filed: Jun. 1, 2012

Related U.S. Application Data
(63) Continuation of application No. PCT/JP2010/071960, filed on Dec. 8, 2010.

(30) Foreign Application Priority Data
Dec. 15, 2009 (JP) 2009-284471

(51) Int. Cl. G01J 1/42; G01N 33/49; H01L 27/146; G01N 21/64; G01J 5/10

(52) U.S. Cl. 250/338.1; 250/216; 250/372; 250/458.1; 250/208.1

(57) ABSTRACT

An optical control device is provided in a scanning optical device that applies light emitted from a light source to an observation target as spot light, and detects return light from the observation target while scanning the spot light, and includes an irradiation section that applies white light and special light to the observation target, an irradiation time control section that performs a control process so that an irradiation time of the special light is longer than an irradiation time of the white light, and a light detection section that detects first return light from the observation target when the white light for which the irradiation time is controlled has been applied to the observation target, and detects second return light from the observation target when the special light for which the irradiation time is controlled has been applied to the observation target.

### CROSS REFERENCE TO RELATED APPLICATION

This application is a continuation of International Patent Application No. PCT/JP2010/71960, having an international filing date of Dec. 8, 2010, which designated the United States, the entirety of which is incorporated herein by reference. Japanese Patent Application No. 2009-284471 filed on Dec. 15, 2009 is also incorporated herein by reference in its entirety.

### BACKGROUND

The present invention relates to an optical control device, a control device, an optical scope, and the like.

A light guide, a forceps channel, a CCD, an air/water supply channel, and the like are normally provided on the end of an insertion section of an endoscopic scope in order to display an image of the object on a monitor. In this case, a burden imposed on the patient and the doctor increases due to an increase in the size of the insertion section of the endoscopic scope. An endoscopic scope may be configured so that the medical examination capability can be improved by switching a light source between a normal light source and a special light source. However, it is difficult to simultaneously display a normal light image and a special light image at the same timing.

In order to deal with the above problems, JP-A-2003-535659 proposes technology that applies light emitted from an RGB laser or another light source to the object in a spot-like shape through an optical fiber while quickly and sequentially switching the light source, and detects return light from the object to form an image. Since JP-A-2003-535659 utilizes a thin optical fiber, the size of the endoscopic scope can be reduced. Moreover, since a normal light image and a special light image can be formed at the same time, the medical examination capability can be improved.

### SUMMARY

According to one aspect of the invention, there is provided an optical control device that is provided in a scanning optical device that applies light emitted from a light source to an observation target as spot light that is applied in a spot-like shape, and detects return light from the observation target while scanning the spot light, the optical control device comprising:

an irradiation section that applies white light and special light to the observation target, the special light being light within a specific wavelength band;

an irradiation time control section that performs a control process so that an irradiation time of the special light is longer than an irradiation time of the white light;

a light detection section that detects first return light from the observation target when the white light for which the irradiation time is controlled has been applied to the observation target, and detects second return light from the observation target when the special light for which the irradiation time is controlled has been applied to the observation target; and

an emission control section,

the irradiation section acquiring the white light from a normal light source that emits the white light, acquiring the special light from a special light source that emits the special light, and scanning a scan target area including the observation target using the white light acquired from the normal light source and the special light acquired from the special light source,

the emission control section controlling an emission timing of the normal light source and the special light source so that the irradiation time of the special light is longer than the irradiation time of the white light, and

the light detection section detecting the first return light and the second return light from the observation target when the irradiation section scans the scan target area.

### BRIEF DESCRIPTION OF THE DRAWINGS

FIG. 1 illustrates a configuration example of a scanning optical device.

FIG. 2 illustrates the spectral characteristics of a normal light source.

FIG. 3 illustrates the spectral characteristics of a special light source (NBI).

FIG. 4 illustrates an example of the emission timing of a normal light source and a special light source.

FIG. 5 illustrates another example of the emission timing of a normal light source and a special light source.

FIG. 6 illustrates a configuration example of an emission control section.

FIG. 7 illustrates a configuration example of an irradiation section.

FIG. 8 illustrates an example of the optical fiber scan direction.

FIG. 9 illustrates an example of an irradiation spot and an emission light source during dot sequential scanning.

FIG. 10 illustrates another example of the optical fiber scan direction.

FIG. 11 illustrates yet another example of the optical fiber scan direction.

FIG. 12 illustrates a configuration example of a light detection section and an image processing section.

FIG. 13 illustrates a configuration example of a first image construction section.

FIG. 14 illustrates a configuration example of a second image construction section.

FIG. 15 illustrates a configuration example of a first interpolation section.

FIG. 16 illustrates a configuration example of a second interpolation section.

FIG. 17 is a view illustrating the configuration of a raster scan image.

FIG. 18 is a view illustrating a bilinear interpolation process.

FIG. 19 illustrates the spectral characteristics of a special light source (AFI).

FIG. 20 illustrates a configuration example of a light detection section (AFI).

FIG. 21 illustrates another configuration example of a scanning optical device.

FIG. 22 illustrates another configuration example of an irradiation section.

FIG. 23 illustrates another configuration example of an irradiation section.

FIGS. 24A, 24B, and 24C illustrate a configuration example of a rotary filter.

FIG. 25 is a view illustrating an insertion section of an endoscope having a forceps channel.

### DESCRIPTION OF EXEMPLARY EMBODIMENTS

According to one aspect of exemplary embodiments of the invention, there is provided an optical control device that is provided in a scanning optical device that applies light emitted from a light source to an observation target as spot light that is applied in a spot-like shape, and detects return light from the observation target while scanning the spot light, the optical control device including an irradiation section that applies white light and special light to the observation target, the special light being light within a specific wavelength band, an irradiation time control section that performs a control process so that an irradiation time of the special light is longer than an irradiation time of the white light, and a light detection section that detects first return light from the observation target when the white light for which the irradiation time is controlled has been applied to the observation target, and detects second return light from the observation target when the special light for which the irradiation time is controlled has been applied to the observation target.

According to one aspect of exemplary embodiments of the invention, the irradiation time of the special light is set to be longer than the irradiation time of the white light, and the return light is detected. This makes it possible to improve the brightness of an image corresponding to the specific wavelength band, and generate a clear image.

According to another aspect of exemplary embodiments of the invention, there is provided a scanning optical device that applies light emitted from a light source to an observation target as spot light that is applied in a spot-like shape, and detects return light from the observation target while scanning the spot light, the scanning optical device including an irradiation section that applies white light and special light to the observation target, the special light being light within a specific wavelength band, an irradiation time control section that performs a control process so that an irradiation time of the special light is longer than an irradiation time of the white light, and a light detection section that detects first return light from the observation target when the white light for which the irradiation time is controlled has been applied to the observation target, and detects second return light from the observation target when the special light for which the irradiation time is controlled has been applied to the observation target, the irradiation section applying the white light acquired from a normal light source that emits the white light, and applying the special light acquired from a special light source that emits the special light.

According to this aspect of exemplary embodiments of the invention, the white light is acquired from the normal light source, and the special light is acquired from the special light source. The irradiation time of the special light is set to be longer than the irradiation time of the white light, and the return light is detected. This makes it possible to implement a scanning optical device that can improve the brightness of an image corresponding to the specific wavelength band, and can generate a clear image.

According to another aspect of exemplary embodiments of the invention, there is provided a scanning optical device that applies light emitted from a light source to an observation target as spot light that is applied in a spot-like shape, and detects return light from the observation target while scanning the spot light, the scanning optical device including an irradiation section that applies white light and special light to the observation target, the special light being light within a specific wavelength band, an irradiation time control section that performs a control process so that an irradiation time of the special light is longer than an irradiation time of the white light, and a light detection section that detects first return light from the observation target when the white light for which the irradiation time is controlled has been applied to the observation target, and detects second return light from the observation target when the special light for which the irradiation time is controlled has been applied to the observation target, the irradiation section acquiring the white light by applying a first filter that allows the white light to pass through to light emitted from a single light source, and acquiring the special light by applying a second filter that allows the special light to pass through to the light emitted from the single light source.

According to this aspect of exemplary embodiments of the invention, the white light and the special light are acquired using a single white light source and a plurality of filters. The irradiation time of the special light is set to be longer than the irradiation time of the white light, and the return light is detected. This makes it possible to implement a scanning optical device that can improve the brightness of an image corresponding to the specific wavelength band, and can generate a clear image.

Exemplary embodiments of the invention are described below. Note that the following exemplary embodiments do not in any way limit the scope of the invention laid out in the claims. Note also that all of the elements described in connection with the following exemplary embodiments should not necessarily be taken as essential elements of the invention.

### 1. First Embodiment

An outline of a method according to a first embodiment of the invention is described below. When using a method that simultaneously acquires a normal light image and a special light image to observe a lesion area, since a lesion area is displayed in the special light image in a color differing from that of the peripheral area (e.g., a lesion such as epidermoid cancer is displayed in brown when using narrow band imaging), the visibility of the lesion area is improved as compared with observation using normal light. However, since the wavelength band of the special light is narrow, and the intensity of the special light is low as compared with observation using noitual light, a dark image that is difficult to observe is obtained using the special light.

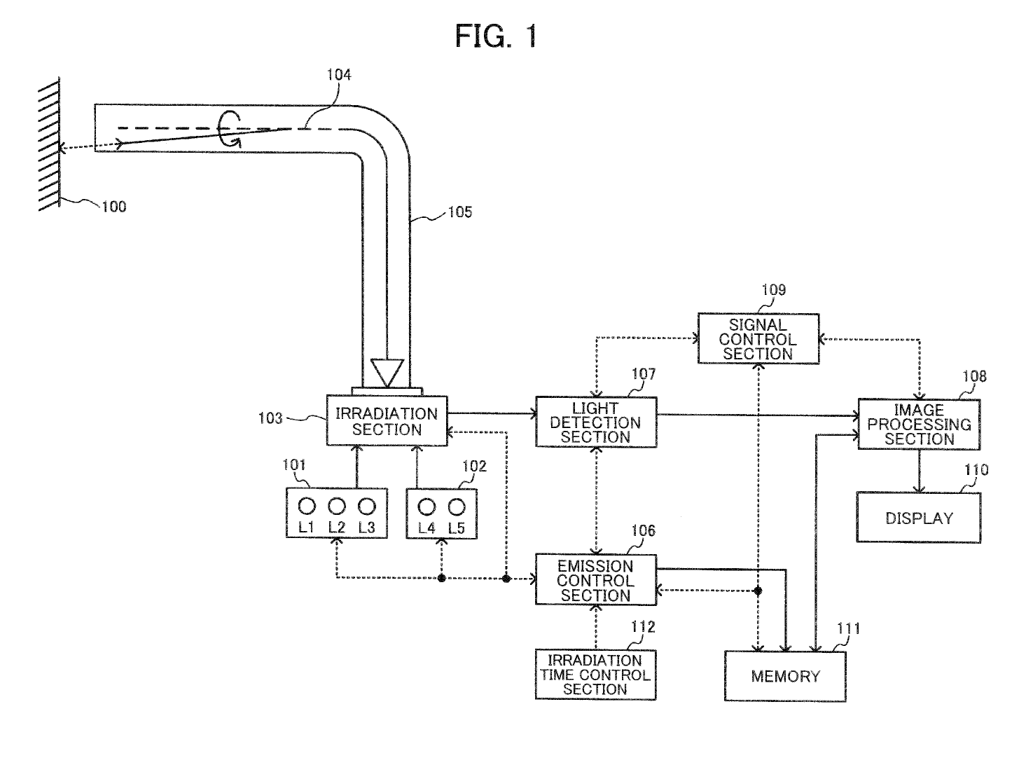

Several aspects of the invention propose a method that acquires a special light image that is bright and contains only a small amount of noise by increasing the irradiation time of the special light as compared with the irradiation time of the normal light. More specifically, an irradiation time control section 112 (see FIG. 1) performs a control process so that the irradiation time of the special light is longer than the irradiation time of the normal light. An emission control section 106 performs the actual emission control process, and emission from a normal light source 101 and a special light source 102 is controlled based on a signal output from the emission control section 106.

FIG. 4 illustrates a specific emission timing. FIG. 4 illustrates the emission timing of the normal light (L1, L2, and L3) and the emission timing of the special light (L4 and L5). Specifically, the control process is performed so that the emission time of light sources L4 and L5 is longer than the emission time of light sources L1, L2, and L3. This makes it possible to improve the brightness of the special light image, and acquire a special light image that is bright and contains only a small amount of noise. The details of the method are described in connection with the first embodiment.

Note that the system configuration is not limited to that illustrated in FIG. 1. A light source section and the like may be configured in a different way. The details of a modification are described in connection with a second embodiment.

FIG. 1 illustrates a configuration example according to the first embodiment. An optical control device (scanning optical device) that observes an object 100 includes a normal light source 101, a special light source 102, an irradiation section 103, an optical fiber 104, an insertion section 105, an emission control section 106, a light detection section 107, an image processing section 108, a signal control section 109, a display 110, a memory 111, and an irradiation time control section 112. Note that the configuration of the optical control device is not limited to the configuration illustrated in FIG. 1. Various modifications may be made, such as omitting some of the elements or adding other elements.

Since the optical control device may be applied to endoscopy, the insertion section 105 is formed in the shape of a pipe that is elongated and can be curved so that the insertion section 105 can be inserted into a body. The optical fiber 104 is inserted into the insertion section 105, and extends from one end to the other end of the insertion section 105. The irradiation section 103 is connected to the insertion section 105. The light detection section 107 receives an optical signal from the irradiation section 103, and transmits the optical signal to the image processing section 108. The image processing section 108 is connected to the display 110. The emission control section 106 is bidirectionally connected to the normal light source 101, the special light source 102, the irradiation section 103, the light detection section 107, the image processing section 108, and the memory 111. The memory 111 is connected to the image processing section 108. The signal control section 109 is bidirectionally connected to the light detection section 107 and the image processing section 108. The irradiation time control section 112 is connected to the emission control section 106.

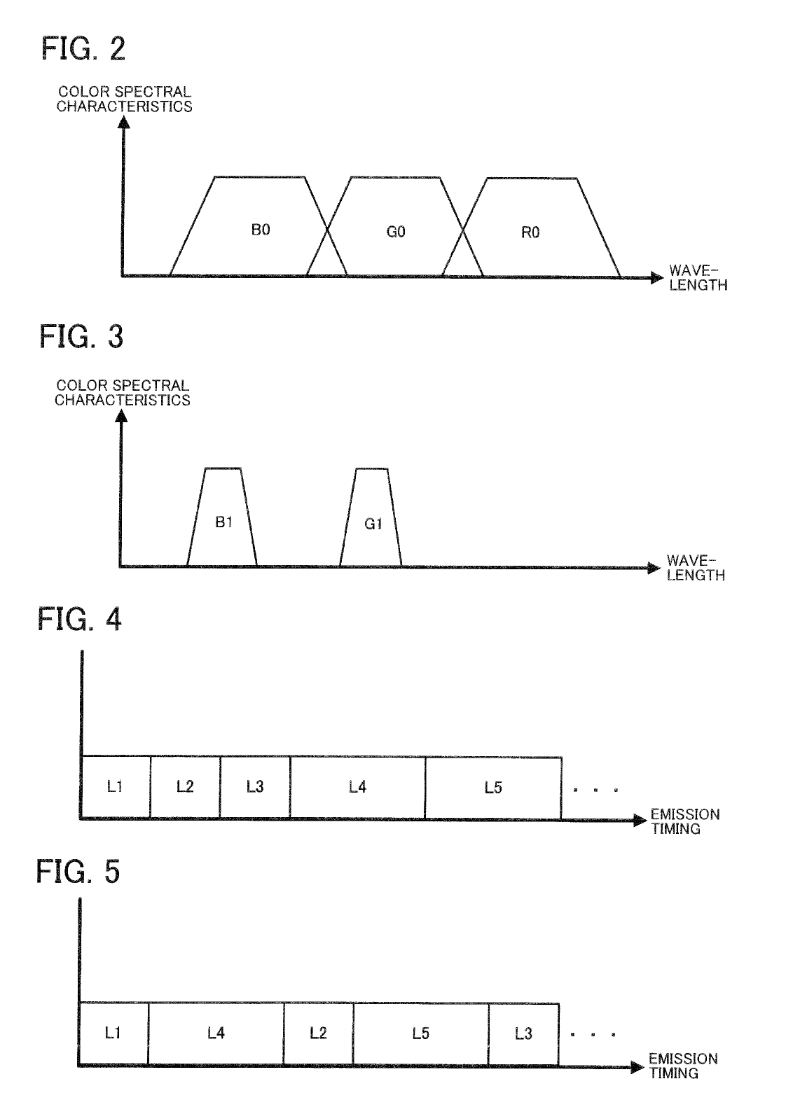

A light source emission control process and the flow of an optical signal and an image signal in FIG. 1 are described below. The noiuial light source 101 includes three monochromatic LED light sources L1, L2, and L3. Each monochromatic LED light source respectively has specific spectral characteristics. The monochromatic LED light source L1 corresponds to spectral characteristics R0 (580 to 700 nm), the monochromatic LED light source L2 corresponds to spectral characteristics G0 (480 to 600 nm), and the monochromatic LED light source L3 corresponds to spectral characteristics B0 (400 to 500 nm) (see FIG. 2). The monochromatic LED light source L1 emits red light, the monochromatic LED light source L2 emits green light, and the monochromatic LED light source L3 emits blue light. White light is obtained by synthesizing light emitted from each monochromatic LED light source. A normal light image is formed by return light obtained when the monochromatic LED light sources L1, L2, and L3 emit light.

The special light source 102 includes two monochromatic LED light sources L4 and L5. Each monochromatic LED light source respectively has specific spectral characteristics. The monochromatic LED light source L4 corresponds to spectral characteristics G1 (530 to 550 nm), and the monochromatic LED light source L5 corresponds to spectral characteristics B1 (390 to 445 nm) (see FIG. 3). In the field of endoscopic diagnosis, light having the narrow-band spectral characteristics G1 and light having the narrow-band spectral characteristics B1 that are easily absorbed by hemoglobin in blood are applied to tissue to form a special light image (NBI image) in which capillaries and a minute mucous membrane pattern in the mucous membrane surface layer are enhanced. The NBI image is effective for diagnosis of esophagus cancer, large bowel cancer, gastric cancer, and the like. The following description is given taking an example of narrow band imaging (NBI) that utilizes special light formed by light having the spectral characteristics G1 and light having the spectral characteristics B1, unless otherwise specified. Note that the special light is not limited to special light in the NBI mode. Light within another wavelength band (e.g., AFI) may also be used.

In the first embodiment, each monochromatic LED light source sequentially and repeatedly emits light at a given emission timing in order from L1→L2→L3→L4→L5 (see FIG. 4) or L1→L4→L2→L5→L3 (see FIG. 5) under control of the emission control section 106, and the light emitted from each monochromatic light source is transmitted to the irradiation section 103. More specifically, the irradiation time control section 112 adjusts the emission timing of the emission control section 106 so that the emission time of the special light sources L4 and L5 is longer than the emission time of the normal light sources L1, L2, and L3 (see FIGS. 4 and 5).

Note that the irradiation spot must remain unchanged when each light source emits light. Therefore, it is necessary to increase the holding time at the irradiation spot (or reduce the scan speed) when the special light source L4 or L5 emits light. Since it is unnecessary to frequently change the holding time when using the method illustrated in FIG. 4 as compared with the method illustrated in FIG. 5, the method illustrated in FIG. 4 can be implemented by a simple mechanical control process. In particular, the method illustrated in FIG. 4 is advantageous when performing a dot sequential process since the light source is switched corresponding to each spot.

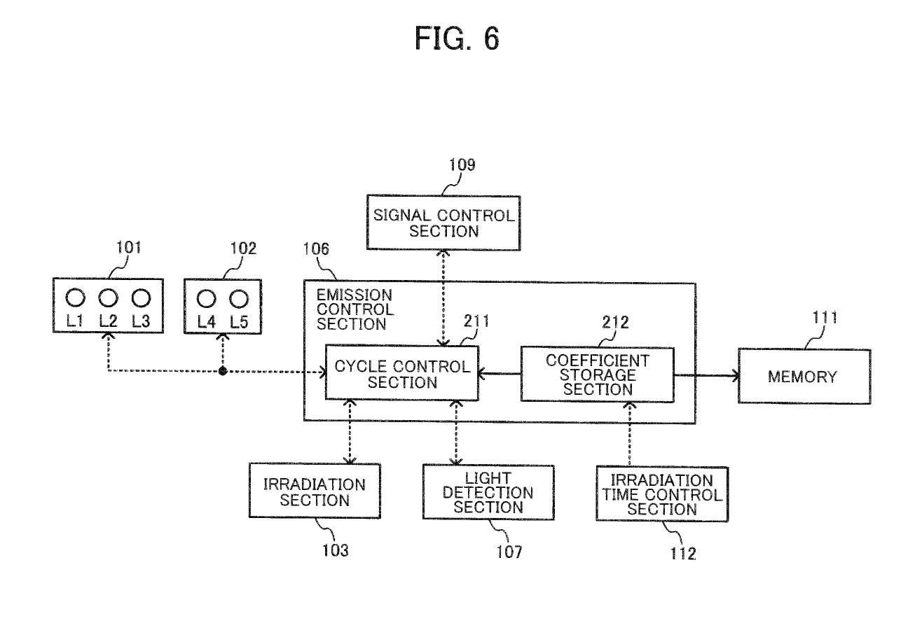

FIG. 6 illustrates an example of the configuration of the emission control section 106. The emission control section 106 includes a cycle control section 211 and a coefficient storage section 212. The cycle control section 211 is bidirectionally connected to the normal light source 101, the special light source 102, the irradiation section 103, the light detection section 107, and the signal control section 109. The coefficient storage section 212 is connected to the memory 111 and the cycle control section 211. The irradiation time control section 112 is connected to the coefficient storage section 212.

The coefficient storage section 212 stores an emission time coefficient that causes the emission time of the special light sources L4 and L5 to be longer than the emission time of the normal light sources L1, L2, and L3 under control of the irradiation time control section 112. More specifically, the coefficient storage section 212 stores a normal light emission time coefficient F1 (ns) and a special light emission time coefficient F2 (ns) (F1<F2), and transmits the emission time coefficient F1 and the emission time coefficient F2 to the cycle control section 211. The cycle control section 211 causes the normal light source 101 and the special light source 102 to sequentially and repeatedly emit light in order from L1→L2→L3→L4→L5 or L1→L4→L2→L5→L3 at time intervals indicated by the emission time coefficient F1 and the emission time coefficient F2. For example, the cycle control section 211 causes the monochromatic light source L1 included in the normal light source 101 to be repeatedly turned ON (F1 (ns))→OFF (F1+F1+F2+F2 (ns))→ON→OFF. Emission information (emission timing and emission cycle) is transmitted to the irradiation section 103, the light detection section 107, and the image processing section 108.

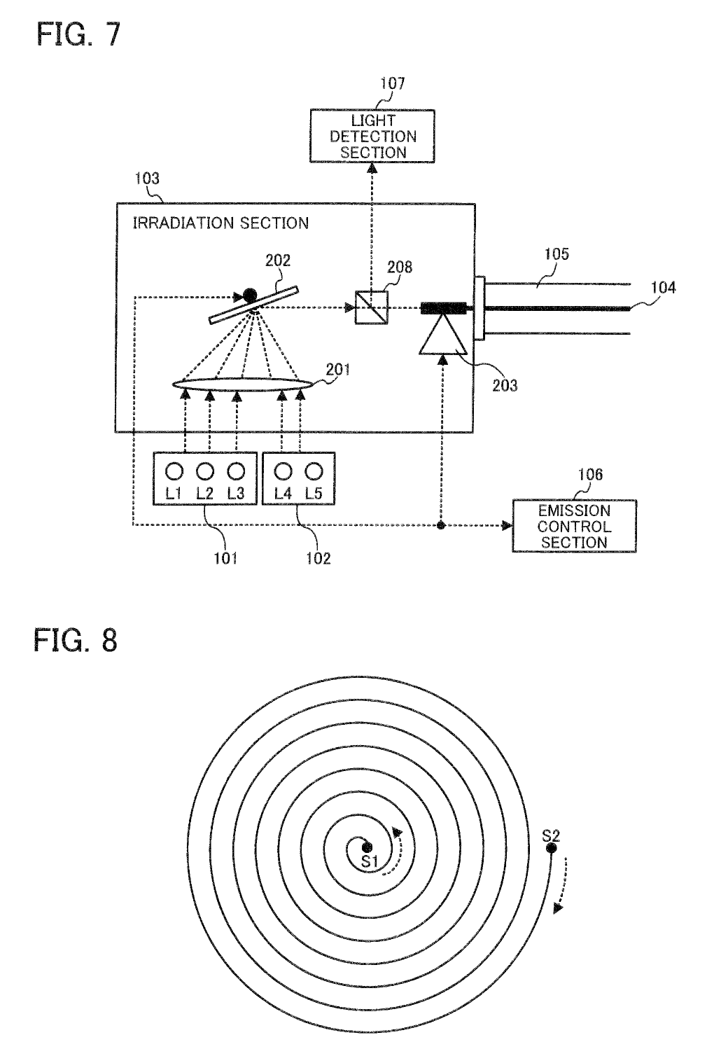

FIG. 7 illustrates an example of the configuration of the irradiation section 103. The irradiation section 103 includes a condenser lens 201, an adjustment mirror 202, a scan control section 203, and a half mirror 208. Light emitted from the normal light source 101 and the special light source 102 enters the condenser lens 201. The light that has entered the condenser lens 201 is incident on the half mirror 208 due to the adjustment mirror 202. The insertion section 105 is connected to the irradiation section. The optical fiber 104 receives the light emitted from the light source through the half mirror 208, and transmits return light from the object 100 to the irradiation section 103. The scan control section 203 is connected to the optical fiber 104. The adjustment mirror 202 and the scan control section 203 are bidirectionally connected to the emission control section 106.

Light emitted from the monochromatic LED light sources L1, L2, and L3 included in the normal light source and the monochromatic LED light sources L4 and L5 included in the special light source sequentially enters the irradiation section 103 at given time intervals based on the above emission timing under control of the emission control section 106. The angle of the adjustment mirror 202 can be adjusted around the center of the adjustment mirror 202. The angle of the adjustment mirror 202 is appropriately adjusted under control of the emission control section 106 corresponding to the type of monochromatic light source that emits light that enters the irradiation section 103. More specifically, the angle of the adjustment mirror 202 is adjusted so that light reflected by the adjustment mirror 202 necessarily enters the optical fiber 104 through the half mirror 208. The normal light source 101 and the special light source 102 thus sequentially and repeatedly emit light having specific spectral characteristics in order from L1→L2→L3→L4→L5 or L1→L4→L2→L5→L3 at given time intervals corresponding to the emission timing, and the light emitted from each light source enters the optical fiber 104. Each monochromatic light that has entered the optical fiber 104 is applied to the object 100 through the optical fiber 104.

A scan method is described below. The scan control section 203 vibrates the optical fiber 104 under control of the emission control section 106, and spirally scans the end of the optical fiber 104 that is positioned at the end of the insertion section 105 around the axis of the optical fiber 104. As illustrated in FIG. 8, the scan control section 203 spirally scans the end of the optical fiber 104 from a start point (center) S1 to an end point S2, for example.

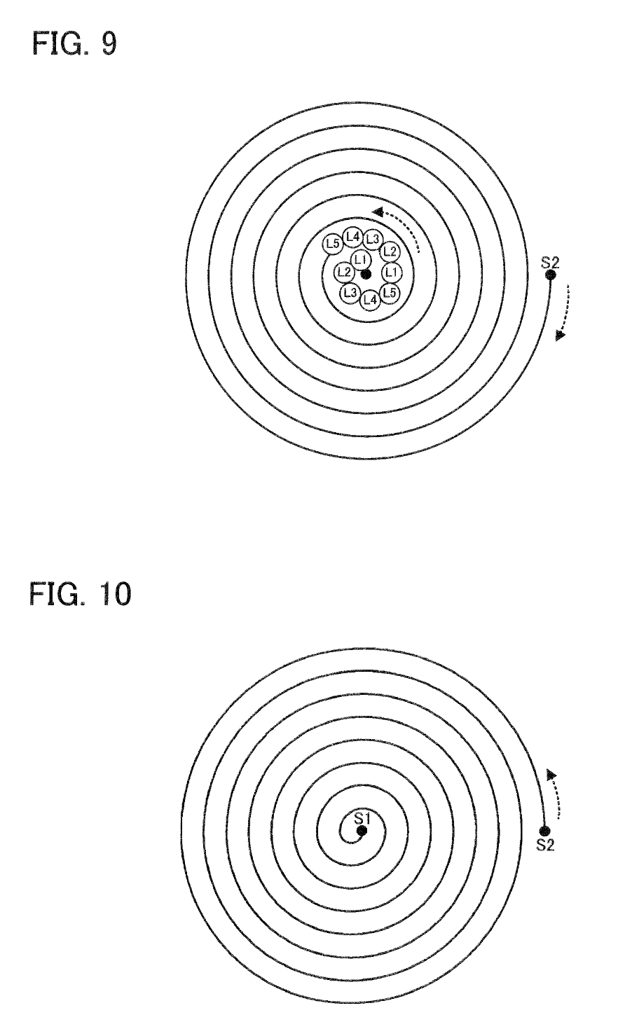

In the first embodiment, the irradiation spot is moved corresponding to the emission timing during a scan. For example, when the scan control section 203 vibrates the optical fiber 104, and scans the end of the optical fiber 104 from the start point S1 to the end point S2 in a state in which the normal light source 101 and the special light source 102 repeatedly emit light in order from L1→L2→L3→L4→L5 (see FIG. 9), the emission timing is controlled so that the given emission time interval corresponds to the irradiation time at one irradiation spot. In this case, one irradiation spot corresponds to one pixel of an image formed by the subsequent process. A two-dimensional image is formed by return light obtained by scanning the end of the optical fiber 104 from the start point S1 to the end point S2.

A method that sequentially applies monochromatic light from the normal light sources L1, L2, and L3 and the special light sources L4 and L5 corresponding to each irradiation spot while scanning the end of the optical fiber 104 from the start point S1 to the end point S2 is referred to as “dot sequential scanning”. The irradiation time control section 112 adjusts the emission timing of the emission control section 106. More specifically, the irradiation time control section 112 adjusts the emission timing of the emission control section 106 so that the emission time of the special light sources L4 and L5 is longer than the emission time of the normal light sources L1, L2, and L3. Therefore, the irradiation time at one irradiation spot when the special light source emits light is longer than that when the normal light source emits light. In this case, the scan control section 203 reduces the scan speed of the optical fiber when the special light source emits light so that the holding time at one irradiation spot is longer than that when the normal light source emits light.

Although a configuration in which one monochromatic light source emits light at one irradiation spot has been described above, another configuration may also be employed. For example, the scan control section 203 may control the scan process so that the normal light sources L1, L2, and L3 and the special light sources L4 and L5 sequentially emit light at one irradiation spot, and the optical fiber is moved to the next irradiation spot after acquiring each return light.

In this case, each monochromatic light source emits light at a single irradiation spot. The irradiation time of the special light monochromatic light source is set to be longer than that of the normal light monochromatic light source in the same manner as described above. Since each monochromatic light source emits light at a single irradiation spot, the holding time at each irradiation spot increases (i.e., the total scan time increases). Therefore, the number of images obtained per unit time decreases, so that the temporal resolution (video performance) deteriorates as compared with normal dot sequential scanning. However, since information corresponding to each light source can be acquired at each irradiation spot, the resolution of the image can be improved.

It is also possible to employ a scan method that causes only one monochromatic light source to emit light while spirally scanning the end of the optical fiber 104 from the start point S1 to the end point S2. This scan method is referred to as “frame sequential scanning”. In this case, the entire area is scanned using one monochromatic light source among the normal light sources L1, L2, and L3 and the special light sources L4 and L5, and return light is acquired at each irradiation spot over the entire area. The entire area is then scanned using another monochromatic light source.

In this case, the irradiation time control section 112 adjusts the emission timing of the emission control section 106 so that the emission time of the special light sources L4 and L5 is longer than the emission time of the normal light sources L1, L2, and L3 in the same manner as in the case of using dot sequential scanning. Therefore, the irradiation time at one irradiation spot when the special light source emits light is longer than that when the normal light source emits light. The scan control section 203 reduces the scan speed of the optical fiber when the special light source emits light so that the holding time at each irradiation spot over the entire area is longer than that when the normal light source emits light.

The following description is given taking dot sequential scanning as an example unless otherwise specified.

The spiral scan direction is not limited to that illustrated in FIG. 8. In FIG. 8, the entire area is spirally scanned from the inner side to the outer side (S1→S2), and then scanned in the reverse direction (i.e., a direction indicated by an arrow) (S2→S1). As illustrated in FIG. 10, the entire area may be scanned from the outer side to the inner side (S2→S1) in the same scan direction (i.e., a direction indicated by an arrow) as that when the entire area is scanned from the inner side to the outer side (S1→S2). In this case, it is possible to continue the scan process without changing the scan speed and the scan direction at the end point S2, so that the mechanical control process is simplied.

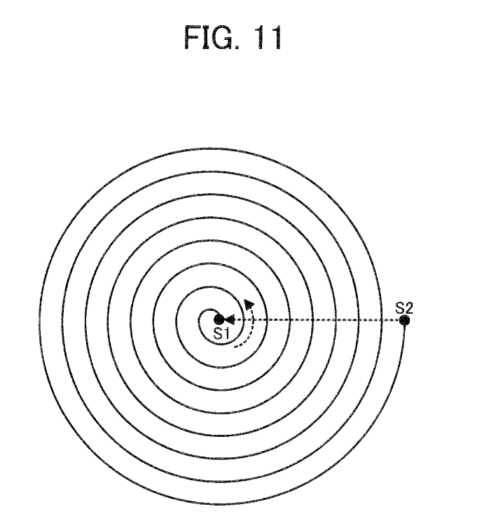

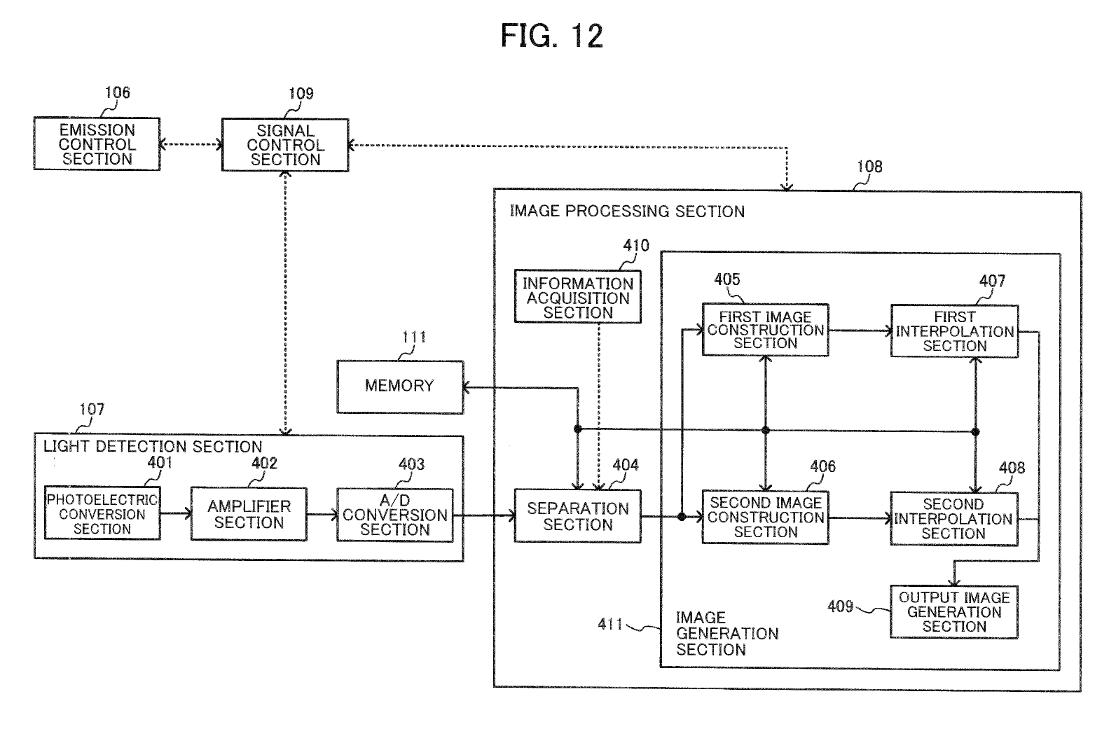

As illustrated in FIG. 11, when the entire area has been spirally scanned from the inner side to the outer side (S1→S2), the optical fiber may be linearly (see arrow) returned to the start point S1 from the end point S2 under control of the scan control section 203, and the entire area may be scanned again from the inner side to the outer side (S1→S2).

Note that coordinate information about each irradiation spot and information about the type of light and the order of emission are transmitted to the memory 111 under control of the emission control section 106. In the first embodiment, the coordinate information about each spot (corresponding to a pixel of an image) is indicated by (x, y) since a two-dimensional image is generated. Note that x is the coordinate value of the two-dimensional image in the horizontal direction, and y is the coordinate value of the two-dimensional image in the vertical direction.

The emission timing is controlled as described below. The irradiation section 103 causes monochromatic light emitted from the monochromatic LED light sources L1, L2, and L3 that forms white light and monochromatic light emitted from the monochromatic LED light sources L4 and L5 that forms special light to be sequentially and repeatedly transmitted to the end of the optical fiber 104 that extends through the insertion section 105 corresponding to the emission timing, and applied to the object 100. The optical fiber 104 receives return light (monochromatic light) from the object 100 at each irradiation spot, and transmits each return light to the light detection section 107 connected to the rear end of the optical fiber 104. A given emission switch time interval of each monochromatic light source (L1, L2, L3, L4, and L5) is set to T1, an elapsed time until light emitted from each monochromatic light source is applied to the object 100 through the irradiation section 103 and the optical fiber 104 is set to T2, and an elapsed time until return light from the object 100 is detected by the light detection section 107 through the optical fiber 104 is set to T3. The emission timing is controlled so that the following expression (1) is satisfied.

T1≧T2+T3 (1)

Specifically, the emission interval is controlled to be equal to or longer than the time elapsed until return light from the object 100 is detected by the light detection section 107 after the light source has emitted light. This makes it possible to control the emission interval so that another light source does not emit light when light emitted from the current light source or return light from the object (observation target) is being transmitted through the optical fiber. Specifically, since two or more types of light do not simultaneously enter the optical fiber, it is possible to observe the object 100 using a single optical fiber while preventing a collision between the optical signals.

The details of the light detection section 107 and the image processing section 108 are described below. Return light from the object 100 enters the irradiation section 103 through the optical fiber 104, and enters the light detection section 107 through the half mirror 208. A signal output from the light detection section 107 is transmitted to the image processing section 108.

FIG. 12 illustrates an example of the configuration of the light detection section 107 and the image processing section 108. The light detection section 107 includes a photoelectric conversion section 401, an amplifier section 402, and an A/D conversion section 403. The image processing section 108 includes a separation section 404, an information acquisition section 410, and an image generation section 411. The image generation section 411 includes a first image construction section 405, a second image construction section 406, a first interpolation section 407, a second interpolation section 408, and an output image generation section 409. Note that the configuration of the light detection section 107 and the image processing section 108 is not limited to the configuration illustrated in FIG. 12. Various modifications may be made, such as omitting some of the elements or adding other elements.

The photoelectric conversion section 401 is connected to the separation section 404 through the amplifier section 402 and the A/D conversion section 403. The separation section 404 is connected to the first interpolation section 407 through the first image construction section 405. The separation section 404 is also connected to the second interpolation section 408 through the second image construction section 406. The first interpolation section 407 and the second interpolation section 408 are connected to the output image generation section 409. The memory 111 is connected to the separation section 404, the first image construction section 405, the second image construction section 406, the first interpolation section 407, and the second interpolation section 408. The signal control section 109 is bidirectionally connected to each section included in the light detection section 107 and the image processing section 108. The emission control section 106 is bidirectionally connected to the signal control section 109. The first interpolation section 407 and the second interpolation section 408 are connected to the output image generation section 409. The information acquisition section 410 is connected to the separation section 404.

In the first embodiment, the photoelectric conversion section 401 performs a photoelectric conversion process using return light corresponding to each irradiation spot detected by the light detection section 107 under control of the signal control section 109, and generates a charge signal so that one pixel corresponds to one irradiation spot. The amplifier section 402 amplifies the charge signal generated by the photoelectric conversion section 401. The A/D conversion section 403 converts the charge signal amplified by the amplifier section 402 into a digital monochromatic image signal, and transmits the digital monochromatic image signal to the separation section 404.

The separation section 404 separates the digital monochromatic image signal based on the type of light source during a scan that corresponds to the irradiation spot and is read from the memory 111 under control of the signal control section 109. More specifically, the separation section 404 transmits a digital monochromatic image signal Rd0 (red band), Gd0 (green band), or Bd0 (blue band) to the first image construction section 405 when the emission light source during a scan is the normal light source L1, L2, or L3, and transmits a digital monochromatic image signal Gd1 or Bd1 to the second image construction section 406 when the emission light source during a scan is the special light source L4 or L5.

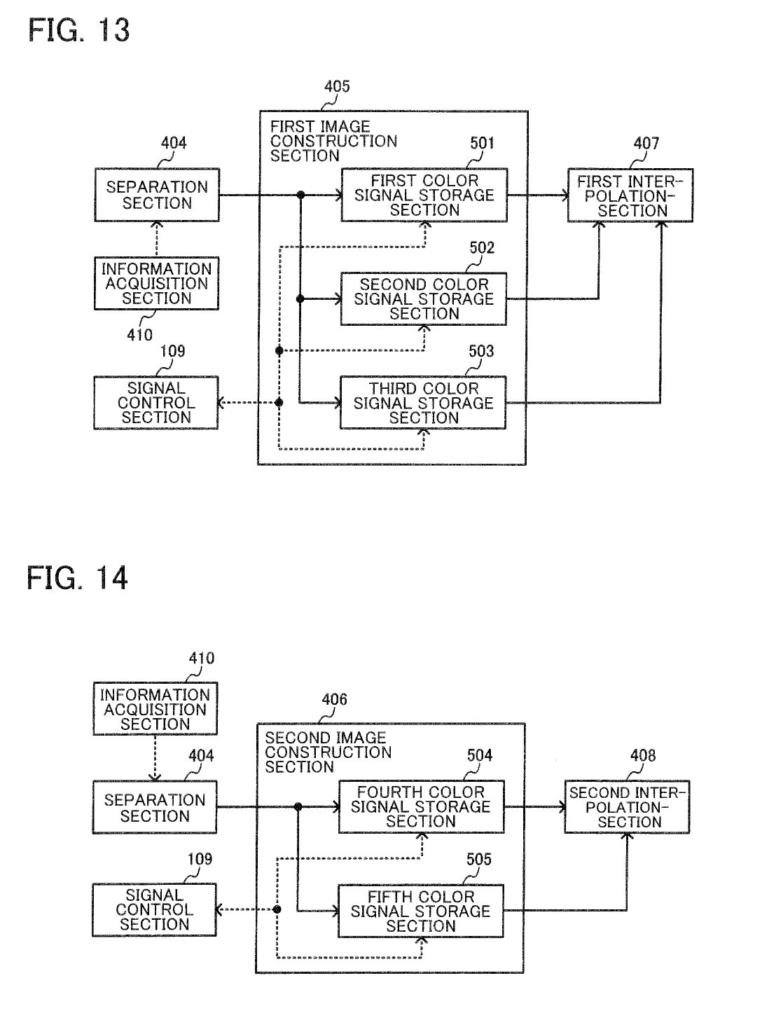

FIG. 13 illustrates an example of the configuration of the first image construction section 405. The first image construction section 405 includes a first color signal storage section 501, a second color signal storage section 502, and a third color signal storage section 503. The separation section 404 is connected to the first color signal storage section 501, the second color signal storage section 502, and the third color signal storage section 503. The first color signal storage section 501, the second color signal storage section 502, and the third color signal storage section 503 are connected to the first interpolation section 407. The signal control section 109 is bidirectionally connected to the first color signal storage section 501, the second color signal storage section 502, and the third color signal storage section 503.

The separation section 404 transmits the digital monochromatic image signal Rd0 (red band) that corresponds to return light when the normal light source L1 has emitted light to the first color signal storage section 501, transmits the digital monochromatic image signal Gd0 (green band) that corresponds to return light when the normal light source L2 has emitted light to the second color signal storage section 502, and transmits the digital monochromatic image signal Bd0 (blue band) that corresponds to return light when the normal light source L3 has emitted light to the third color signal storage section 503 under control of the signal control section 109. Each digital monochromatic image signal is linked to the coordinate information (x, y).

When the entire area has been scanned using the optical fiber 104 under control of the signal control section 109, the digital monochromatic image signal Rd0 (red band) corresponding to the entire area stored in the first color signal storage section 501, the digital monochromatic image signal Gd0 (green band) corresponding to the entire area stored in the second color signal storage section 502, and the digital monochromatic image signal Bd0 (blue band) corresponding to the entire area stored in the third color signal storage section 503 are transmitted to the first interpolation section 407.

When performing frame sequential scanning, each of the normal light sources L1, L2, and L3 emits light over the entire area (i.e., the entire area is scanned three times) under control of the signal control section 109, and the digital monochromatic image signal Rd0 (red band) corresponding to the entire area stored in the first color signal storage section 501, the digital monochromatic image signal Gd0 (green band) corresponding to the entire area stored in the second color signal storage section 502, and the digital monochromatic image signal Bd0 (blue band) corresponding to the entire area stored in the third color signal storage section 503 are transmitted to the first interpolation section 407.

FIG. 14 illustrates an example of the configuration of the second image construction section 406. The second image construction section 406 includes a fourth color signal storage section 504 and a fifth color signal storage section 505. The separation section 404 is connected to the fourth color signal storage section 504 and the fifth color signal storage section 505. The fourth color signal storage section 504 and the fifth color signal storage section 505 are connected to the second interpolation section 408. The signal control section 109 is bidirectionally connected to the fourth color signal storage section 504 and the fifth color signal storage section 505.

The separation section 404 transmits the digital monochromatic image signal Gd1 (narrow-band color) that corresponds to return light when the special light source L4 has emitted light to the fourth color signal storage section 504, and transmits the digital monochromatic image signal Bd1 (narrow-band color) that corresponds to return light when the special light source L5 has emitted light to the fifth color signal storage section 505 under control of the signal control section 109. Each digital monochromatic image signal is linked to the coordinate information (x, y). The digital monochromatic image signal Gd1 (narrow-band color) stored in the fourth color signal storage section 504, and the digital monochromatic image signal Bd1 (narrow-band color) stored in the fifth color signal storage section 505 are transmitted to the second interpolation section 408.

The digital monochromatic image signals Rd0, Gd0, and Bd0 correspond to a scan over the entire area using the normal light source, and the digital monochromatic image signals Gd1 and Bd1 correspond to a scan over the entire area using the special light source. The signals Rd0, Gd0, Bd0, Gd1, and Bd1 respectively indicate a two-dimensional spiral image corresponding to the scan direction of the optical fiber.

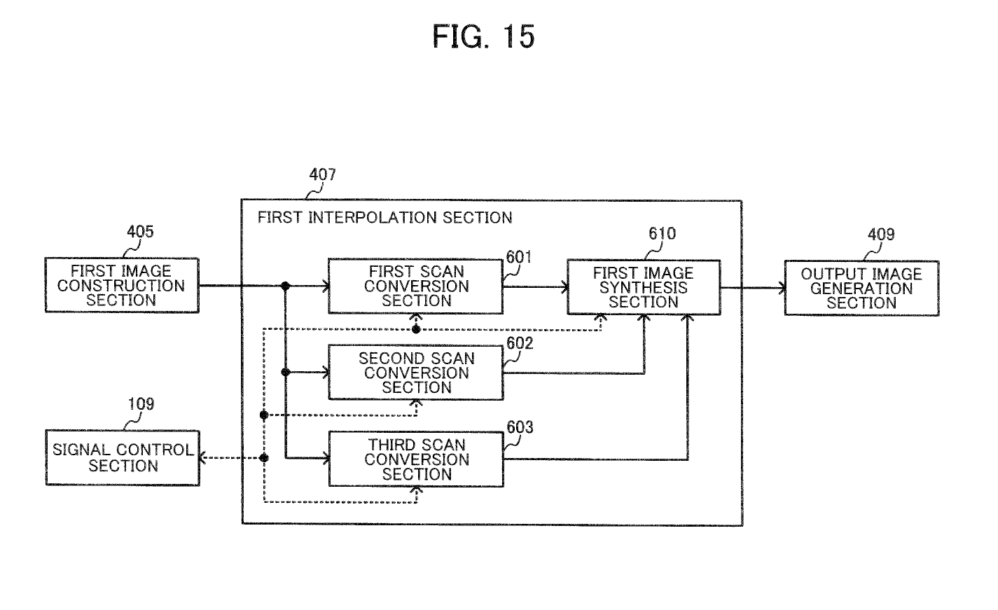

FIG. 15 illustrates an example of the configuration of the first interpolation section 407. The first interpolation section 407 includes a first scan conversion section 601, a second scan conversion section 602, a third scan conversion section 603, and a first image synthesis section 610. The first image construction section 405 is connected to the first scan conversion section 601, the second scan conversion section 602, and the third scan conversion section 603. The first scan conversion section 601, the second scan conversion section 602, and the third scan conversion section 603 are connected to the first image synthesis section 610. The first image synthesis section 610 is connected to the output image generation section 409. The signal control section 109 is bidirectionally connected to the first scan conversion section 601, the second scan conversion section 602, the third scan conversion section 603, and the first image synthesis section 610. Note that the configuration of the first interpolation section is not limited to the configuration illustrated in FIG. 15. Various modifications may be made, such as omitting some of the elements illustrated in FIG. 15.

A spiral Rd0 monochromatic image input from the first image construction section 405 is transmitted to the first scan conversion section 601, a spiral Gd0 monochromatic image input from the first image construction section 405 is transmitted to the second scan conversion section 602, and a spiral Bd0 monochromatic image input from the first image construction section 405 is transmitted to the third scan conversion section 603 under control of the signal control section 109. Since the Rd0 monochromatic image input to the first scan conversion section 601, the Gd0 monochromatic image input to the second scan conversion section 602, and the Bd0 monochromatic image input to the third scan conversion section 603 are two-dimensional spiral images, each pixel is displaced from the original position. Therefore, it is necessary to correct the distortion by performing geometrical transformation using a shape correction function shown by the following expression (2).

V′([x′],[y′])=f(V([x],[y])) (2)

where, V(x, y) is the pixel value of the spiral image, x is the coordinate value of the spiral image in the horizontal direction, y is the coordinate value of the spiral image in the vertical direction, V′(x′, y′) is the pixel value of the raster scan image, x′ is the coordinate value of the raster scan image in the horizontal direction, and y′ is the coordinate value of the raster scan image in the vertical direction.

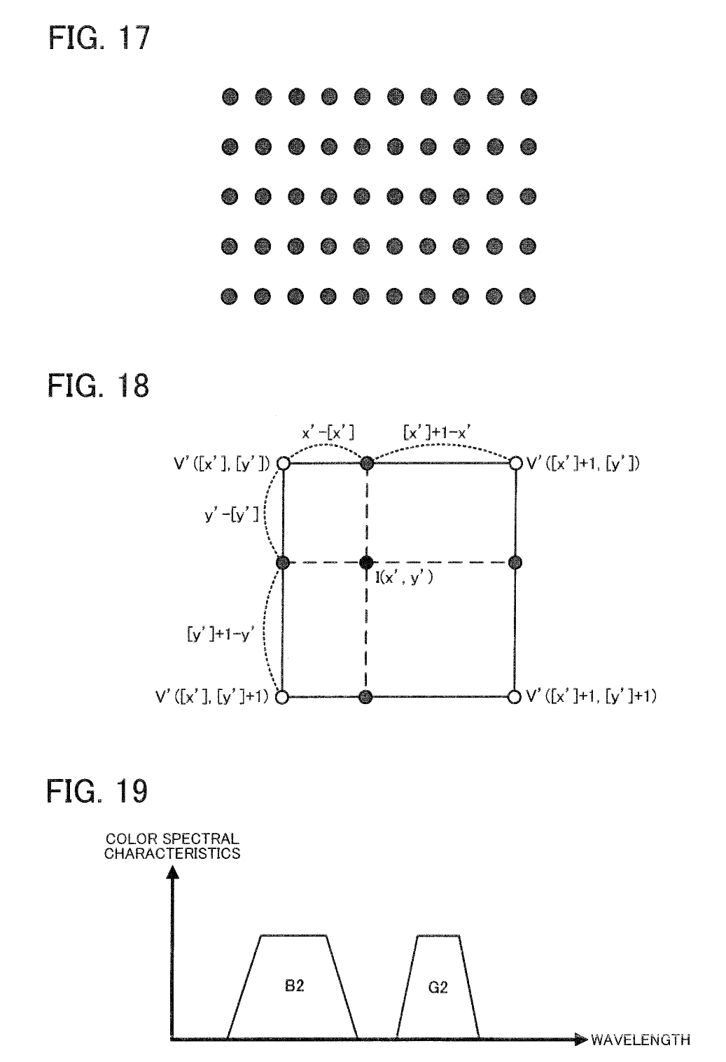

Since the image obtained by geometrical transformation has a dead pixel, it is necessary to perform an interpolation process on the image in order to obtain the target two-dimensional raster scan image illustrated in FIG. 17. In the first embodiment, the pixel value I(x′, y′) at the target position is calculated by the following expression (3) using the pixel values of four peripheral points based on a known bilinear interpolation method (see FIG. 18).

I(x′,y′)=([x′]+1−x′)([y′]+1−y′)V′([x′],[y′])+([x′]+1−x′)(y′−[y′])V′([x′],[y′]+1)+(x′−[x′])([y′]+1−y′)V′([x′]+1,[y′])+(x′−[x′])(y′−[y′])V′([x′]+1,[y′]+1) (3)

The two-dimensional raster scan image illustrated in FIG. 17 is obtained by the interpolation process using the expression (3).

The Rd0 monochromatic image that has been converted into a raster scan image is transmitted from the first scan conversion section 601 to the first image synthesis section 610, the Gd0 monochromatic image that has been converted into a raster scan image is transmitted from the second scan conversion section 602 to the first image synthesis section 610, and the Bd0 monochromatic image that has been converted into a raster scan image is transmitted from the third scan conversion section 603 to the first image synthesis section 610.

The first image synthesis section 610 synthesizes a 3-channel RGB normal light image based on the following expression (4) using the Rd0 monochromatic image, the Gd0 monochromatic image, and the Bd0 monochromatic image (that have been converted into a raster scan image) under control of the signal control section 109, and transmits the RGB normal light image to the output image generation section 409.

Rchv=Rd0_{—}v

Gch_{—}v=Gd0_{—}v

Bch_{—}v=Bd0_{—}v (4)

where, Rch_v is the pixel value of the R channel of the RGB normal light image, Gch_v is the pixel value of the G channel of the RGB normal light image, Bch_v is the pixel value of the B channel of the RGB normal light image, Rd0_v is the pixel value of the Rd0 monochromatic image, Gd0_v is the pixel value of the Gd0 monochromatic image, and Bd0_v is the pixel value of the Bd0 monochromatic image.

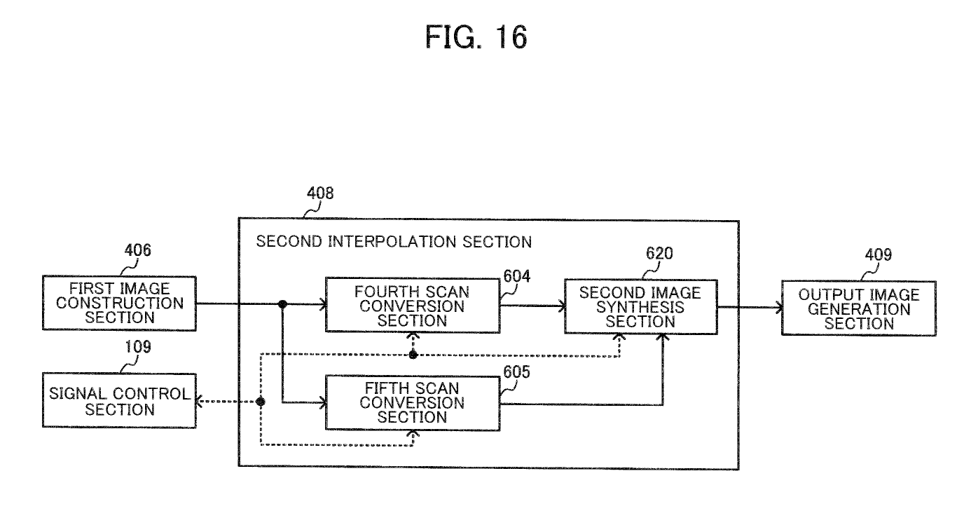

FIG. 16 illustrates an example of the configuration of the second interpolation section 408. The second interpolation section 408 includes a fourth scan conversion section 604, a fifth scan conversion section 605, and a second image synthesis section 620. The second image construction section 406 is connected to the fourth scan conversion section 604 and the fifth scan conversion section 605. The fourth scan conversion section 604 and the fifth scan conversion section 605 are connected to the second image synthesis section 620. The second image synthesis section 620 is connected to the output image generation section 409. The signal control section 109 is bidirectionally connected to the fourth scan conversion section 604, the fifth scan conversion section 605, and the second image synthesis section 620.

A spiral Gd1 monochromatic image input from the second image construction section 406 is transmitted to the fourth scan conversion section 604, and a spiral Bd1 monochromatic image input from the second image construction section 406 is transmitted to the fifth scan conversion section 605 under control of the signal control section 109. Since the Gd1 monochromatic image input to the fourth scan conversion section 604 and the Bd1 monochromatic image input to the fifth scan conversion section 605 are two-dimensional spiral images, the Gd1 monochromatic image and the Bd1 monochromatic image are converted into a two-dimensional raster scan image (see FIG. 17) using the shape correction function shown by the expression (2) and the bilinear interpolation process shown by the expression (3).

The second image synthesis section 620 synthesizes an NBI special light image (NBI pseudo-color image) based on the following expression (5) using the Gd1 monochromatic image and the Bd1 monochromatic image (that have been converted into a raster scan image) under control of the signal control section 109, and transmits the NBI special light image to the output image generation section 409.

Rch_{—}v=p1*Bd1_{—}v

Gch_{—}v=p2*Bd1_{—}v

Bch_{—}v=p3*Gd1_{—}v (5)

where, Rch_v is the pixel value of the R channel of the NBI special light image, Gch_v is the pixel value of the G channel of the NBI special light image, Bch_v is the pixel value of the B channel of the NBI special light image, Bd1_v is the pixel value of the Bd1 image, Gd1_v is the pixel value of the Gd1 image, and p1, p2, and p3 are given coefficients.

The output image generation section 409 performs image processing (e.g., noise reduction process, white balance correction process, color conversion process, and grayscale transformation process) on each pixel of the 3-channel RGB normal light image and the NBI special light image (that have been converted into a raster scan image) transmitted from the first image synthesis section 610 and the second image synthesis section 620, and transmits the RGB normal light image and the NBI special light image subjected to image processing to the display 110.

As described above, the normal light sources L1, L2, and L3 and the special light sources L4 and L5 (monochromatic light sources) are caused to sequentially and repeatedly emit light at the given emission timing while vibrating the optical fiber 104. Light is applied to the object through the optical fiber 104, and return light is sequentially and repeatedly received to obtain a normal light image and a special light image (NBI image) at the same time. Since the emission timing is controlled so that the emission time of the special light sources L4 and L5 is longer than the emission time of the normal light sources L1, L2, and L3, the irradiation time at one irradiation spot when the special light source emits light is longer than that when the normal light source emits light, so that the sensitivity of the special light image is improved. This makes it possible to improve the diagnostic capability for esophagus cancer, large bowel cancer, gastric cancer, and the like.

Although an example in which the NBI (narrow band imaging) image is formed by causing the narrow-band special light source that corresponds to the wavelength band of light absorbed by hemoglobin in blood to emit light has been described above, special light sources having the spectral characteristics of excitation light (390 to 470 nm) used to observe intrinsic fluorescence produced by a fluorescent substance (e.g., collagen) and light (540 to 560 nm) that is absorbed by hemoglobin in blood (see FIG. 19) may be caused to emit light, and an AFI (autofluorescence imaging) special light image may be formed based on the return light. AFI is a technique that applies narrow-band excitation light to an in vivo object, and detects intrinsic fluorescence produced by the in vivo object due to the excitation light to form a special light image. AFI is effective for diagnosing epidermoid cancer of a bronchial tube, early-stage esophagus cancer, and a colorectal neoplastic lesion area.

The AFI special light image is basically formed in the same manner as the NBI special light image, except for the following points.

Specifically, the monochromatic LED light source L4 included in the special light source 102 has transmittance characteristics G2 (540 to 560 nm), and the monochromatic LED light source L5 included in the special light source 102 has transmittance characteristics B2 (390 to 470 nm).

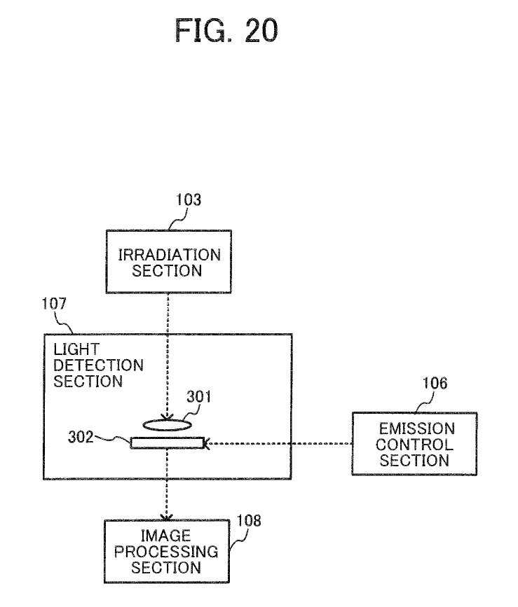

FIG. 20 illustrates an example of the configuration of the light detection section 107. The light detection section 107 includes a condenser lens 301 and a barrier filter 302. The barrier filter 302 is bidirectionally connected to the emission control section 106.

The barrier filter 302 can be moved under control of the emission control section 106. When causing the monochromatic LED light source L5 to emit excitation light under control of the emission control section 106, the barrier filter 302 (transmittance characteristics: 470 to 690 nm) is moved and inserted into the optical path of return light at each irradiation spot that enters from the irradiation section 103 so that intrinsic fluorescence (490 to 625 nm) passes through, while return light (390 to 470 nm) of the excitation light is blocked. When causing the monochromatic LED light source other than the monochromatic LED light source L5 to emit light, the barrier filter 302 is removed from the optical path of return light transmitted through the optical fiber under control of the emission control section 106. The barrier filter 302 is thus inserted into or removed from the optical path of return light transmitted through the optical fiber corresponding to the timing at which the monochromatic LED light sources L1, L2, L3, L4, and L5 sequentially and repeatedly emit light under control of the emission control section 106.

The second image synthesis section 620 synthesizes a 3-channel AFI special light image based on the following expression (6) using a Gd2 (narrow-band) monochromatic image and a Bd2 (narrow-band) monochromatic image (that have been converted into a raster scan image) under control of the signal control section 109, and transmits the AFI special light image to the output image generation section 409. The Gd2 image is an image formed when the monochromatic LED light source LA (spectral characteristics: 540 to 560 nm) has emitted light. The Bd2 image is an image formed due to intrinsic fluorescence (spectral characteristics: 490 to 625 nm) produced by in vivo tissue when the monochromatic LED light source L5 (spectral characteristics: 390 to 470 nm) has emitted light.

Rch_{—}v=Gd2_{—}v

Gch_{—}v=Bd2_{—}v

Bch_{—}v=Gd2_{—}v (6)

where, Rch_v is the pixel value of the R channel of the AFI special light image, Gch_v is the pixel value of the G channel of the AFI special light image, Bch_v is the pixel value of the B channel of the AFI special light image, Gd2_v is the pixel value of the return light Gd2 image, and Bd2_v is the pixel value of the return light Bd2 image.

Alternatively, a monochromatic LED light source having spectral characteristics of infrared light (790 to 820 nm) may be provided as the monochromatic LED light source L4 included in the special light source 102, a monochromatic LED light source having spectral characteristics of infrared light (905 to 970 nm) may be provided as the monochromatic LED light source L5 included in the special light source 102, infrared light may be applied to the object after intravenously injecting indocyanine green (ICG) that easily absorbs infrared light, and an IRI (infrared imaging) special light image may be formed based on the return light. In this case, since a vessel or the blood flow in a deep area of a mucous membrane that is difficult to observe visually can be highlighted, the depth of gastric cancer invasion or the therapeutic strategy can be determined, or esophageal variceal sclerotherapy can be effectively performed.

As described above, white light and special light within a specific wavelength band are acquired based on light emitted from the light source while vibrating the optical fiber corresponding to the given emission timing, and sequentially and repeatedly applied to the object through the optical fiber. The normal light image can be formed by detecting the white light that has returned from the object, the special light image can be formed by detecting the special light that has returned from the object, and the normal light image and the special light image can be displayed on the display at the same time. This makes it possible to improve the diagnostic capability. Moreover, since the emission timing is controlled so that the irradiation time of special light is longer than the irradiation time of white light, the sensitivity of the special light image can be improved.

The first embodiment may be applied to an optical control device that is provided in a scanning optical device (e.g., endoscope device) that applies spot light to an observation target, and detects return light while scanning spot light. In the first embodiment, the optical control device corresponds to a functional block that includes at least the irradiation section 103, the irradiation time control section 112, and the light detection section 107. The irradiation section 103 applies white light and special light to the observation target. The irradiation time control section 112 performs a control process so that the irradiation time of the special light is longer than the irradiation time of the white light. The light detection section 107 detects first return light from the observation target when the white light has been applied to the observation target, and detects second return light from the observation target when the special light has been applied to the observation target.

Note that the term “spot light” used herein refers to light that is applied to the observation target in a spot-like shape. The term “special light” used herein refers to light within a specific wavelength band. For example, special light used for narrow band imaging (NBI) refers to light within a wavelength band of 390 to 445 nm and 530 to 550 nm.

The above configuration makes it possible to set the irradiation time of the special light to be longer than the irradiation time of the white light when applying the white light and the special light in a spot-like shape. Therefore, since the irradiation quantity (intensity per unit time×irradiation time) of the special light can be increased as compared with the normal light, it is possible to prevent a situation in which the brightness of an image (second image in a broad sense) corresponding to the specific wavelength band is insufficient, so that a clear image can be generated.

The irradiation section 103 may acquire the white light from a normal light source that emits the white light, and may acquire the special light from a special light source that emits the special light.

According to this configuration, since the white light can be acquired from the normal light source, and the special light can be acquired from the special light source, it is possible to acquire the white light and the special light in a simple and intuitive way. Moreover, since it is unnecessary to use a filter or the like, the configuration of the irradiation section 103 can be simplified.

The optical control device may include the emission control section 106. The emission control section 106 controls the emission timing of the normal light source and the special light source so that the irradiation time of the special light is longer than the irradiation time of the white light.

This makes it possible to control emission of the normal light source and the special light source so that the time set (controlled) by the irradiation time control section 112 (i.e., the irradiation time of the special light is longer than the irradiation time of the white light) is implemented.

The normal light source 101 may include first to Nth (N is an integer equal to or larger than 2) monochromatic light sources that respectively emit first to Nth monochromatic lights that form white light. The emission control section 106 may perform a control process so that the first to Nth monochromatic light sources sequentially emit light, and the irradiation section 103 may sequentially acquire and apply the first to Nth monochromatic lights. The first to Nth monochromatic lights may be R color light, G color light, and B color light.

This makes it possible to utilize a light source that emits a plurality of monochromatic lights that form white light as the normal light source. The plurality of monochromatic lights that form white light can be sequentially applied to the observation target by causing the plurality of light sources to sequentially emit light.

The plurality of monochromatic lights that form white light may be R color light, G color light, and B color light. In this case, the normal light source can be implemented using light sources that are generally used and emit familiar light.

The special light source 102 may include (N+1)th to Mth (M is an integer that satisfies “M>N+1”, and N is an integer) monochromatic light sources that respectively emit (N+1)th to Mth monochromatic lights that form the special light. The emission control section 106 may perform a control process so that the (N+1)th to Mth monochromatic light sources sequentially emit light, and the irradiation section 103 may sequentially acquire and apply the (N+1)th to Mth monochromatic lights.

This makes it possible to utilize a light source that emits a plurality of monochromatic lights that form special light as the special light source. The plurality of monochromatic lights that form special light can be sequentially applied to the observation target by causing the plurality of light sources to sequentially emit light. When implementing narrow band imaging (NBI), for example, a light source that emits light within a wavelength band of 390 to 445 nm, and a light source that emits light within a wavelength band of 530 to 550 nm may be used.

The irradiation section 103 may scan a scan target area using the white light and the special light. The light detection section 107 may detect first return light (return light that corresponds to the white light applied to the observation target in a narrow sense (reflected light in a narrower sense)) and second return light (return light that corresponds to the special light applied to the observation target in a narrow sense (reflected light or fluorescence in a narrower sense)) from the observation target when the irradiation section 103 scans the scan target area. Note that the term “scan target area” used herein refers to an area that includes the observation target, and corresponds to a screen displayed on the display 110.

This makes it possible to scan the scan target area, and detect return light. Optical information sufficient to form a screen can be sequentially acquired by thus scanning the scan target area and detecting return light, even if only optical information corresponding to one pixel can be acquired by applying spot light.

When the entirety of the scan target area has been scanned by the irradiation section 103 using one of the white light and the special light, the emission control section 106 may cause the light source that emits the other of the white light and the special light to emit light, and the irradiation section 103 may scan the entirety of the scan target area using the other of the white light and the special light.

The details of the above process are described below taking an example in which the entirety of the scan target area is scanned counterclockwise from the start point S1 to the end point S2, and then scanned clockwise from the end point S2 to the start point S1 (see FIG. 8). In this case, the entirety of the scan target area is scanned from the start point S1 to the end point S2 using one (e.g., white light) of the white light and the special light. The white light is continuously applied during this period. The entirety of the scan target area is then scanned from the end point S2 to the start point S1 using the other (e.g., special light) of the white light and the special light. The special light is continuously applied during this period. The entirety of the scan target area is then scanned using one of the white light and the special light, and scanned using the other of the white light and the special light.

This makes it possible to implement frame sequential scanning. The term “frame sequential scanning” refers to a scan method that scans the entirety of the scan target area using one light, and then scans the entirety of the scan target area using another light. Since only a single-color image is obtained by scanning the entire region scan once, it is necessary to scan the entirety of the scan target area P times to form one screen when the number of lights used is P. Therefore, the number of images obtained per unit time decreases, so that the temporal resolution (video performance) decreases as compared with dot sequential scanning. However, since each light has information corresponding to each irradiation spot, the resolution can be increased.

The normal light source 101 may include first to Nth (N is an integer equal to or larger than 2) monochromatic light sources that respectively emit first to Nth monochromatic lights that form white light. When the entirety of the scan target area has been scanned by the irradiation section 103 using ith light, the emission control section 106 may cause the light source that emits (i+1)th light to emit light, and the irradiation section 103 may scan the entirety of the scan target area using the (i+1)th light.

The details of the above process are described below taking the scan method illustrated in FIG. 8. When the first to Nth monochromatic light sources that respectively emit the first to Nth monochromatic lights that form white light are R, G, and B monochromatic light sources, the entirety of the scan target area is scanned from the start point S1 to the end point S2 using the R light source. The entirety of the scan target area is then scanned from the end point S2 to the start point S1 using the G light source, and scanned from the start point S1 to the end point S2 using the B light source. This makes it possible to acquire optical information corresponding to one normal light image. When continuously acquiring an image, the entirety of the scan target area is then scanned from the end point S2 to the start point S1 using the R light source, and scanned from the start point S1 to the end point S2 using the G light source.

This makes it possible to implement frame sequential scanning by sequentially utilizing the light sources that respectively emit a plurality of lights that form white light instead of merely switching light between the white light and the special light.

The special light source 102 may include (N+1)th to Mth (M is an integer that satisfies “M>N+1”, and N is an integer) monochromatic light sources that respectively emit (N+1)th to Mth monochromatic lights that form the special light. When the entirety of the scan target area has been scanned by the irradiation section 103 using jth light, the emission control section 106 may cause the light source that emits (j+1)th light to emit light, and the irradiation section 103 may scan the entirety of the scan target area using the (j+1)th light.

The details of the above process are described below taking the scan method illustrated in FIG. 8. When the (N+1)th to Mth monochromatic light sources that respectively emit the (N+1)th to Mth monochromatic lights that form special light are G1 and B1 monochromatic light sources used for NBI, the entirety of the scan target area is scanned from the start point S1 to the end point S2 using the G1 light source. The entirety of the scan target area is then scanned from the end point S2 to the start point S1 using the B1 light source. This makes it possible to acquire optical information corresponding to one special light image. When continuously acquiring an image, the entirety of the scan target area is then scanned from the start point S1 to the end point S2 using the G1 light source, and scanned from the end point S2 to the start point S1 using the B1 light source.

This makes it possible to implement frame sequential scanning by sequentially utilizing the light sources that respectively emit a plurality of lights that form special light instead of merely switching light between the white light and the special light.

When the irradiation section 103 has applied one of the white light and the special light to one irradiation spot, the emission control section 106 may cause the light source that emits the other of the white light and the special light to emit light, and the irradiation section 103 may apply the other of the white light and the special light to the next irradiation spot.

FIG. 9 illustrates an example when the above configuration is employed. When causing the light sources L1 to L5 (the light sources L1 to L3 correspond to the normal light, and the light sources L4 and L5 correspond to the special light) to sequentially emit light (see FIG. 4), the light source L1 (e.g., R light source) emits light at the first irradiation spot when scanning the entirety of the scan target area from the start point S1 (i.e., the center point of the spiral) (not illustrated in FIG. 9) to the end point S2. The light source L2 (e.g., G light source) then emits light at the next irradiation spot. The light source that emits light and the irradiation spot are similarly changed thereafter so that one light source corresponds to one irradiation spot (i.e., the light source L3 (e.g., B light source) emits light at the next irradiation spot, the light source L4 (e.g., G1 light source) emits light at the next irradiation spot, and the light source L5 (e.g., B1 light source) emits light at the next irradiation spot). When the light source L5 has emitted light, the light source L1 again emits light. This process is repeated until the end point S2 is reached. It is possible to acquire optical information corresponding to one normal light image and one special light image by scanning the entirety of the scan target area from the start point S1 to the end point S2. When continuously acquiring an image, the entirety of the scan target area is scanned again in the same manner as described above using the scan method illustrated in FIG. 8, 10, or 11.

This makes it possible to implement dot sequential scanning. The term “dot sequential scanning” used herein refers to a scan method that sequentially changes light applied to the observation target corresponding to each irradiation spot. When utilizing dot sequential scanning, it is possible to acquire an image corresponding to each color by scanning the entirety of the scan target area once. Therefore, the number of images obtained per unit time increases, so that the temporal resolution increases. On the other hand, since information about each color can be obtained at only one spot among Q spots when the number of colors is Q, dot sequential scanning is inferior to frame sequential scanning in terms of resolution. Whether to use dot sequential scanning or frame sequential scanning is determined depending on the situation. It may be advantageous to employ dot sequential scanning that achieves high video performance when moving the optical scope at a relatively high speed (e.g., when searching a lesion area), and employ frame sequential scanning when the optical scope is moved to only a small extent, and it is desired to give priority to the resolution (e.g., when it is desired to closely observe a lesion area).

The emission control section 106 may include the cycle control section 211 that controls the emission timing of the normal light source and the special light source so that the white light and the special light emit light are alternately emitted in each cycle. The term “cycle” used herein refers to a time within which the normal light source and the special light source respectively emit light once. When the white light and the special light are implemented by a plurality of light sources, the term “cycle” used herein refers to a time within which each light source respectively emits light once.

The above configuration makes it possible to control the emission timing so that the normal light source and the special light source alternately emit light in each cycle. More specifically, the emission timing is controlled as illustrated in FIG. 4 or 5. As illustrated in FIG. 4, the light sources L1, L2, and L3 may be considered to be a single normal light source, the light sources L4 and L5 may be considered to be a single special light source, and the emission timing may be controlled so that the special light source emits light after the normal light source has emitted light. This also falls under the scope of the expression “alternately emit light”. As illustrated in FIG. 5, each color that forms the white light and each color that forms the special light may be alternately emitted.

The term “specific wavelength band” may be a band that is narrower than the wavelength band of white light. Specifically, the specific wavelength band may be the wavelength band of light absorbed by hemoglobin in blood. More specifically, the specific wavelength band may be 390 to 445 nm or 530 to 550 nm.

This makes it possible to implement narrow band imaging (NBI). NBI makes it possible to observe the structure of a surface area of tissue and a blood vessel located in a deep area. A lesion area (e.g., epidermoid cancer) that cannot be easily observed using normal light can be displayed as a brown area or the like by inputting the resulting signal to a given channel (R, G, or B), so that the lesion area can be reliably detected (i.e., a situation in which the lesion area is missed can be prevented). A wavelength band of 390 to 445 nm or 530 to 550 nm is selected from the viewpoint of absorption by hemoglobin and the ability to reach a surface area or a deep area of tissue. Note that the wavelength band is not limited thereto. For example, the lower limit of the wavelength band may decrease by about 0 to 10%, and the upper limit of the wavelength band may increase by about 0 to 10% depending on a variation factor (e.g., experimental results for absorption by hemoglobin and the ability to reach a surface area or a deep area of tissue).

The specific wavelength band may be the wavelength band of excitation light that causes a fluorescent substance to produce fluorescence. More specifically, the wavelength band of fluorescence may be 490 to 625 nm, and the wavelength band of excitation light may be 390 to 470 nm.

This makes it possible to implement autofluorescence imaging (AFI). Intrinsic fluorescence produced by a fluorescent substance (e.g., collagen) can be observed by applying excitation light (390 to 470 nm). In this case, the lesion area can be highlighted in a color differing from that of a normal mucous membrane, so that the lesion area can be reliably detected, for example. A wavelength band of 490 to 625 nm is the wavelength band of fluorescence produced from a fluorescent substance (e.g., collagen) when excitation light is applied. A wavelength band of 390 to 470 nm is the wavelength band of excitation light that causes a fluorescent substance to produce fluorescence. Note that the wavelength band is not limited thereto. For example, the lower limit of the wavelength band may decrease by about 0 to 10%, and the upper limit of the wavelength band may increase by about 0 to 10% depending on a variation factor (e.g., experimental results for the wavelength band of fluorescence produced by a fluorescent substance). A pseudo-color image may be generated by simultaneously applying light within a wavelength band of 540 to 560 nm that is absorbed by hemoglobin.

The specific wavelength band may be the wavelength band of infrared light. More specifically, the specific wavelength band may be 790 to 820 nm or 905 to 970 nm.

This makes it possible to implement infrared imaging (IRI). Information about a blood vessel or a blood flow in a deep area of a mucous membrane that cannot be easily observed visually can be highlighted by intravenously injecting indocyanine green (ICG) (infrared marker) that easily absorbs infrared light, and applying infrared light within the above wavelength band, so that the depth of gastric cancer invasion or the therapeutic strategy can be determined, for example. An infrared marker exhibits maximum absorption within a wavelength band of 790 to 820 nm, and exhibits minimum absorption within a wavelength band of 905 to 970 nm. Note that the wavelength band is not limited thereto. For example, the lower limit of the wavelength band may decrease by about 0 to 10%, and the upper limit of the wavelength band may increase by about 0 to 10% depending on a variation factor (e.g., experimental results for absorption by the infrared marker).

The scanning optical device according to the first embodiment may be a scanning endoscope.

This makes it possible to implement a scanning endoscope that includes the optical control device according to the first embodiment.

The first embodiment may also be applied to a control device that includes the optical control device and the image processing section 108. The image processing section 108 may generate an output image using the first return light and the second return light.

In this case, the optical control device acquires an optical signal, converts the optical signal into an electrical signal, and subjects the electrical signal to an A/D conversion process to acquire a digital signal. The image processing section 108 performs image processing on the acquired digital signal. This makes it possible to display an image in an appropriate format. More specifically, a first image (normal light image in a narrow sense) is generated from the first return light, and a second image (special light image in a narrow sense) is generated from the second return light. The output image is generated from the first image and the second image.

The image processing section 108 may include the information acquisition section 410, the separation section 404, and the image generation section 411. The information acquisition section 410 may acquire light identification information. The separation section 404 may separate return light from the observation target into the first return light and the second return light based on the light identification information. The image generation section 411 may generate the output image based on the first return light and the second return light. The term “light identification information” used herein refers to information that specifies the type of light applied to the observation target. For example, the light identification information specifies whether the light applied to the observation target is the white light or the special light. When the normal light source 101 and the special light source 102 respectively include a plurality of light sources, the light identification information specifies a light source among the plurality of light sources that has emitted light.

This makes it possible to specify the type of light applied to the observation target. Therefore, return light can be appropriately separated into the first return light and the second return light. It is also possible to generate the first image and the second image based on appropriate return light, and generate an appropriate output image.

The light detection section 107 may detect the first return light when the white light has been applied to the observation target, and may detect the second return light when the special light has been applied to the observation target. As illustrated in FIG. 1, return light that corresponds to light emitted from the light sources L1 to L3 is the first return light, and return light that corresponds to light emitted from the light sources L4 and L5 is the second return light.

This makes it possible to clearly link the white light to the first return light, and clearly link the special light to the second return light.

The image generation section 411 may generate the first image based on the first return light, and may generate the second image based on the second return light. The image generation section 411 may generate the output image from the first image and the second image.

This makes it possible to clearly link the first return light to the first image (i.e., link the white light to the first image), and clearly link the second return light to the second image (i.e., link the special light to the second image). This makes it possible to appropriately generate the first image and the second image, and also generate an appropriate output image.

The information acquisition section 410 may acquire the light identification information that specifies whether the light applied to the observation target is monochromatic light among the first to Nth monochromatic lights that form the white light or monochromatic light among the (N+1)th to Mth monochromatic lights that form the special light. The separation section 404 may separate return light into first to Nth return lights and (N+1)th to Mth return lights based on the light identification information.

For example, when the light sources L1 to L5 are provided as illustrated in FIG. 1, the separation section 404 transmits information corresponding to the light sources L1 to L3 to the first image construction section 405, and transmits information corresponding to the light sources L4 and L5 to the second image construction section 406 (see FIG. 12) based on the light identification information. As illustrated in FIGS. 13 and 14, the separation section 404 transmits information corresponding to the light source L1 to the first color signal storage section 501, and transmits information corresponding to the light source L2 to the second color signal storage section 502. The separation section 404 also transmits information corresponding to the light sources L3 to L5 to the corresponding color signal storage sections.

This makes it possible to appropriately separate the return light even when the normal light source includes a plurality of monochromatic light sources that respectively emit a plurality of lights that form the white light, and the special light source includes a plurality of monochromatic light sources that respectively emit a plurality of lights that form the special light.

The light detection section 107 may detect the first to Nth monochromatic return lights when the first to Nth monochromatic lights that form the white light have been applied. The image generation section 411 may generate first to Nth monochromatic images that form the first image based on the first to Nth monochromatic return lights.

This makes it possible to generate the first to Nth monochromatic images from the first to Nth monochromatic return lights. For example, it is possible to generate an R color monochromatic image, a G color monochromatic image, and a B color monochromatic image. The first image (normal light image in a narrow sense) can be generated by inputting these monochromatic images to the R channel, the G channel, and the B channel, respectively.

The light detection section 107 may detect the (N+1)th to Mth monochromatic return lights when the (N+1)th to Mth monochromatic lights that form the special light have been applied. The image generation section 411 may generate (N+1)th to Mth monochromatic images that form the second image based on the (N+1)th to Mth monochromatic return lights.

This makes it possible to generate the (N+1)th to Mth monochromatic images from the (N+1)th to Mth monochromatic return lights. For example, it is possible to generate a G1 color monochromatic image and B1 color monochromatic image used for narrow band imaging. The second image (special light image in a narrow sense) can be generated by inputting these monochromatic images to the R channel, the G channel, and the B channel, respectively.

The irradiation section 103 may spirally apply the spot light. As illustrated in FIG. 12, the image processing section 108 may include the information acquisition section 410 that acquires position information about the spot light, and the image generation section 411 included in the image processing section 108 may include the first interpolation section 407 and the second interpolation section 408. The first interpolation section 407 may convert the format of a first image signal (e.g., image signals corresponding to light emitted from the light sources L1 to L3 in FIG. 1) that corresponds to the first return light separated by the separation section 404 into a raster scan format based on the position information acquired by the information acquisition section 410. Likewise, the second interpolation section 408 may convert the format of a second image signal (e.g., image signals corresponding to light emitted from the light sources L4 and L5 in FIG. 1) that corresponds to the second return light into a raster scan format based on the position information. The term “raster scan format” used herein refers to the image format illustrated in FIG. 17. The first interpolation section 407 and the second interpolation section 408 also perform the bilinear interpolation process illustrated in FIG. 18. The image generation section 411 may generate the first image based on the first image signal that has been converted into the raster scan format, and may generate the second image based on the second image signal that has been converted into the raster scan format. More specifically, the first image synthesis section 610 illustrated in FIG. 15 generates the first image, and the second image synthesis section 620 illustrated in FIG. 16 generates the second image.

This makes it possible to convert the format of the image (i.e., an unnatural image in which the observation target is distorted) obtained by a spiral scan into the raster scan format (see FIG. 17). Since a dead pixel without information may occur when merely correcting the distortion, the dead pixel is interpolated by the bilinear interpolation process or the like. An image can be generated based on the resulting raster scan image signal.

The first embodiment may also be applied to an optical scope that allows the white light applied by the irradiation section included in the optical control device according to the first embodiment to pass through, and transmits return light from the observation target to the light detection section.

The term “optical scope” used herein corresponds to the insertion section 105 illustrated in FIG. 1. Specific examples of the optical scope include an upper gastrointestinal scope, a lower gastrointestinal scope, and the like. Since a specific identification number is assigned to each optical scope, each scope can be identified by storing the identification number in a memory, for example. Since a different scope is used depending on the observation target area, the observed area can be specified by identifying the scope.

The first embodiment may also be applied to a scanning optical device that includes the irradiation section 103, the irradiation time control section 112, and the light detection section 107. The irradiation section 103 may apply white light and special light to the observation target. The irradiation time control section 112 may perform a control process so that the irradiation time of the special light is longer than the irradiation time of the white light. The light detection section 107 may detect first return light from the observation target when the white light has been applied to the observation target, and may detect second return light from the observation target when the special light has been applied to the observation target. The irradiation section may acquire the white light from a normal light source and apply the white light, and may acquire the special light from a special light source and apply the special light. The normal light source may include a plurality of light sources that respectively emit a plurality of lights that form the white light, and the special light source may include a plurality of light sources that respectively emit a plurality of lights that form the special light.

The above configuration makes it possible to set the irradiation time of the special light to be longer than the irradiation time of the white light when applying the white light and the special light in a spot-like shape. Therefore, since the irradiation quantity (intensity per unit time x irradiation time) of the special light can be increased as compared with the normal light, it is possible to implement a scanning optical device (e.g., scanning endoscope in a narrow sense) that can prevent a situation in which the brightness of an image (second image in a broad sense) corresponding to the specific wavelength band is insufficient, and can generate a clear image. In this case, the configuration of the irradiation section 103 can be simplified in an intuitive manner since the white light is acquired from the normal light source, and the special light is acquired from the special light source. Note that the normal light source and the special light source may respectively include a plurality of monochromatic light sources.

### 2. Second Embodiment

FIG. 21 illustrates a configuration example according to the second embodiment. An optical control device that observes an object 100 includes a normal light source 101, an irradiation section 103, an optical fiber 104, an insertion section 105, an emission control section 106, a light detection section 107, an image processing section 108, a signal control section 109, a display 110, a memory 111, and an irradiation time control section 112. Note that the configuration of the optical control device is not limited to the configuration illustrated in FIG. 21. Various modifications may be made, such as omitting some of the elements or adding other elements.

The optical control device according to the second embodiment is basically the same as the optical control device according to the first embodiment. The following description focuses on the differences from the optical control device according to the first embodiment. In the second embodiment, the normal light source 101 emits white light.

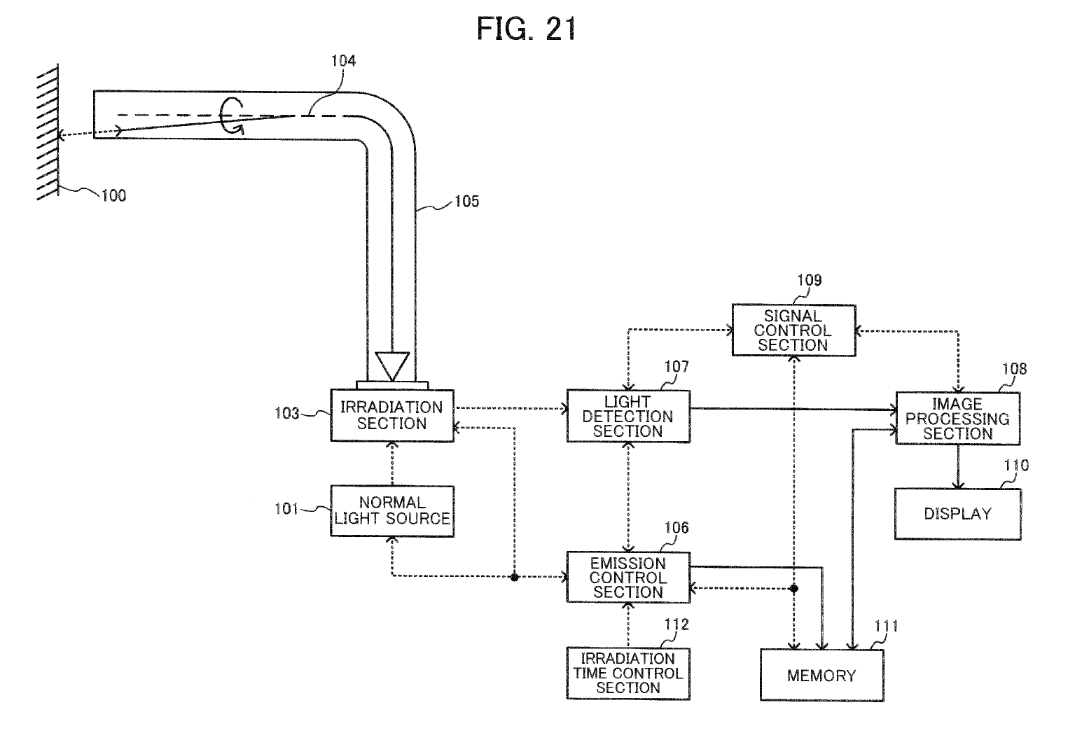

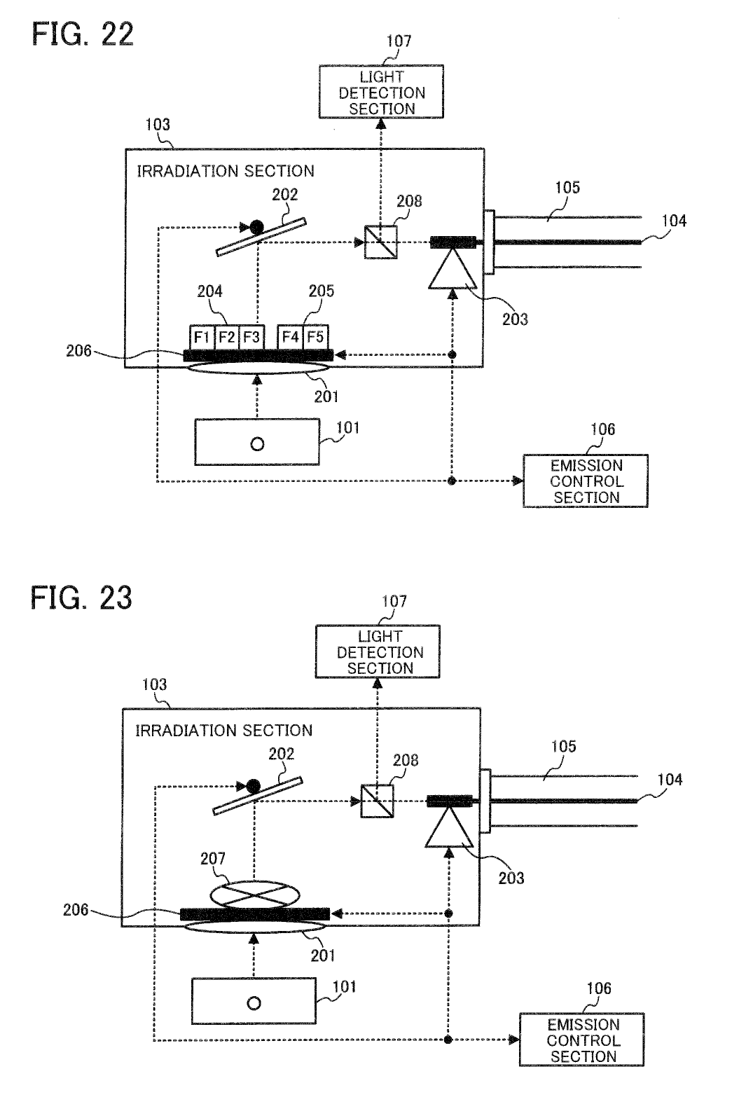

FIG. 22 illustrates an example of the configuration of the irradiation section 103. The irradiation section 103 includes a condenser lens 201, an adjustment mirror 202, a scan control section 203, a first filter 204, a second filter 205, a filter control section 206, and a half mirror 208. Light emitted from the normal light source 101 enters the condenser lens 201. The insertion section 105 is connected to the irradiation section 103. The optical fiber 104 receives the light emitted from the normal light source 101 through the half mirror 208, and transmits return light from the object 100 to the irradiation section 103. The first filter 204 and the second filter 205 are connected to the filter control section 206. The scan control section 203 is connected to the optical fiber 104. The emission control section 106 is bidirectionally connected to the adjustment mirror 202, the scan control section 203, and the filter control section 206. White light emitted from the normal light source 101 enters the condenser lens 201.

The first filter includes filters F1, F2, and F3. The filter F1 has transmittance characteristics that allow light within a wavelength band R0 (580 to 700 nm) to pass through, the filter F2 has transmittance characteristics that allow light within a wavelength band G0 (480 to 600 nm) to pass through, and the filter F3 has transmittance characteristics that allow light within a wavelength band B0 (400 to 500 nm) to pass through (see FIG. 2). Specifically, red light is obtained when the white light emitted from the normal light source 101 has passed through the filter F1, green light is obtained when the white light emitted from the normal light source 101 has passed through the filter F2, and blue light is obtained when the white light emitted from the normal light source 101 has passed through the filter F3. Light that has passed through each filter (F1, F2, and F3) is applied to the object through the optical fiber 104, and a normal light image is formed by return light from the object.

The second filter includes filters F4 and F5. The filter F4 has transmittance characteristics that allow light within a wavelength band G1 (530 to 550 nm) to pass through, and the filter F5 has transmittance characteristics that allow light within a wavelength band B1 (390 to 445 nm) to pass through (see FIG. 3). Light (narrow-band light) that has passed through the filters F4 and F5 is applied to the object through the optical fiber 104, and an NBI special light image is formed by return light from the object.

An AFI special light image can be formed when the filter F4 has transmittance characteristics that allow light within a wavelength band G2 (540 to 560 nm) to pass through, and the filter F5 has transmittance characteristics that allow light within a wavelength band B2 (390 to 470 nm) to pass through. An IRI special light image can be formed when the filter F4 has transmittance characteristics that allow light within a wavelength band (790 to 820 nm) of infrared light to pass through, and the filter F5 has transmittance characteristics that allow light within a wavelength band (905 to 970 nm) of infrared light to pass through.

The normal light source 101 is secured, and the filter control section 206 can be repeatedly moved in the horizontal direction (left→right or right→left). Therefore, white light emitted from the normal light source is sequentially and repeatedly applied to the filters F1, F2, F3, F4, and F5 under control of the emission control section 106. Monochromatic light that has passed through each filter is sequentially transmitted to the optical fiber 104 through the adjustment mirror 202 and the half mirror. Note that the filters F1, F2, F3, F4, and F5 may be arranged in the vertical direction, and white light emitted from the normal light source 101 may be sequentially and repeatedly applied to the filters F1, F2, F3, F4, and F5 while vertically moving the filter control section 206 under control of the emission control section 106.

In the second embodiment, the irradiation time control section 112 performs a control process through the emission control section 106 so that the irradiation time of light applied to the filters F4 and F5 that allow the special light to pass through is longer than the irradiation time of light applied to the filters F1, F2, and F3 that allow the normal light to pass through. The above control process achieves the same emission effect as that obtained when the monochromatic LED light sources L1, L2, L3, L4, and L5 according to the first embodiment sequentially emit light so that the irradiation time of the special light is longer than the irradiation time of the white light.

FIG. 23 illustrates a modification of the configuration of the irradiation section 103. In FIG. 23, the irradiation section 103 includes the condenser lens 201, the adjustment mirror 202, the scan control section 203, the filter control section 206, a rotary filter 207, and the half mirror 208. Light emitted from the normal light source 101 enters the condenser lens 201. The insertion section 105 is connected to the irradiation section. The optical fiber 104 receives the light emitted from the normal light source 101 through the half mirror 208, and transmits return light from the object 100 to the irradiation section 103. The rotary filter 207 is connected to the filter control section 206. The scan control section 203 is connected to the optical fiber 104. The emission control section 106 is bidirectionally connected to the adjustment mirror 202, the scan control section 203, and the filter control section 206.

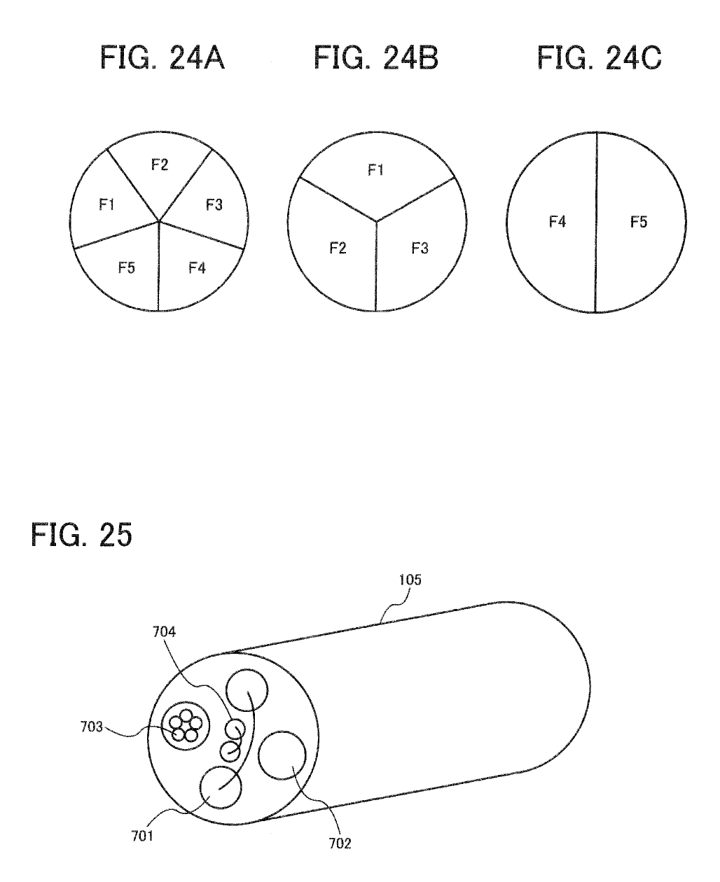

As illustrated in FIG. 24A, the rotary filter includes five monochromatic filters F1, F2, F3, F4, and F5. The transmittance characteristics of the filters F1 to F5 are the same as those described above. Specifically, the rotary filter is formed by combining the first filter and the second filter. The rotary filter is configured so that the area of the filters F4 and F5 corresponding to the special light is larger than the area of the filters F1, F2, and F3 corresponding to white light.

The rotary filter 207 is rotated by controlling the filter control section 206 at a given emission timing under control of the emission control section 106. Therefore, white light emitted from the normal light source 101 is sequentially and repeatedly applied to the filters F1, F2, F3, F4, and F5, and light that has passed through each filter is sequentially and repeatedly transmitted to the optical fiber 104 through the adjustment mirror 202 and the half mirror 208. Since the area of the filters F4 and F5 corresponding to the special light is larger than the area of the filters F1, F2, and F3 corresponding to white light, the above control process achieves the same emission effect as that obtained when light sources sequentially emit light so that the irradiation time of the special light is longer than the irradiation time of the white light.

As illustrated in FIGS. 24B and 24C, a first rotary filter may be formed by combining the filters F1, F2, and F3, and a second rotary filter may be formed by combining the filters F4 and F5. The first rotary filter and the second rotary filter are configured so that the area of the filters F1, F2, and F3 corresponding to the white light is smaller than the area of the filters F4 and F5 corresponding to the special light.

In this case, the first rotary filter and the second rotary filter are rotated by controlling the filter control section 206 at a given emission timing under control of the emission control section 106 while repeatedly applying white light emitted from the normal light source 101 to the first rotary filter and the second rotary filter. This makes it possible to set the emission time of the special light to be longer than the emission time of the white light in the same manner as described above referring to FIG. 24A.

The optical control device may be provided in an endoscopic scope having a forceps channel in order to achieve a reduction in cost. For example, the optical fiber of the optical control device may be inserted into the forceps channel of the endoscopic scope.

FIG. 25 illustrates an example of an endoscopic scope having a forceps channel. The end of an insertion section 105 of the endoscopic scope includes a light guide 701, a forceps channel 702, a CCD 703, and a air/water supply channel 704. For example, the optical fiber 104 is inserted into the forceps channel 702 so that the optical fiber 104 extends between the ends of the forceps channel 702. When using such a configuration, the light guide 701 and the CCD 703 are turned OFF, and the optical fiber is caused to vibrate at given emission timing in the same manner as described above. The white light and the special light are sequentially and repeatedly applied to the observation target through the optical fiber 104 so that the irradiation time of the special light is longer than the irradiation time of the white light, and a normal light image and a special light image are formed using return light from the observation target.

A white light image and a special light image can be generated at the same time by thus inserting the optical fiber into the endoscopic scope having a forceps channel, and sequentially and repeatedly applying the white light and the special light to the observation target. It is also possible to observe the observation target using the white light and the special light when using an endoscopic scope that can deal with only white light and does not allow observation using the special light. This makes it possible to improve the diagnostic capability while achieving a reduction in cost.

According to the second embodiment, a single light source is provided. The irradiation section 103 acquires the white light by applying the first filter that allows the white light to pass through to light emitted from the single light source, and acquires the special light by applying the second filter that allows the special light to pass through to light emitted from the single light source. The irradiation time control section 112 performs a control process so that the application time of the second filter is longer than the application time of the first filter. The first filter corresponds to the first filter 204 illustrated in FIG. 22, and includes a filter that allows light that forms the white light to pass through. The second filter corresponds to the second filter 205 illustrated in FIG. 22, and includes a filter that allows light that forms the special light to pass through.

This makes it possible to acquire the white light and the special light from the single light source. The irradiation time of the special light is set to be longer than the irradiation time of the normal light by changing the application time of each filter. The configuration of the light source section is simplified by utilizing the single light source. Moreover, since it is unnecessary to adjust the adjustment mirror 202 illustrated in FIGS. 22 and 23 corresponding to the type of light, the mechanical control process is facilitated.

The irradiation section 103 may sequentially acquire the white light and the special light by rotating a rotary filter that includes the first filter and the second filter. In this case, the rotary filter is configured so that the size of the second filter is larger than the size of the first filter.

This makes it possible to acquire the normal light image and the special light image using the rotary filter (see FIG. 23). The rotary filter is configured as illustrated in FIG. 24A, for example. Since light to be acquired can be changed by rotating the filter, the mechanical control process is facilitated, and the filter can be quickly switched between the first filter and the second filter as compared with a configuration in which the filters are moved in the horizontal direction or the vertical direction (see FIG. 22).

The second embodiment may also be applied to a scanning optical device that includes the irradiation section 103, the irradiation time control section 112, and the light detection section 107. The irradiation section 103 may apply white light and special light to the observation target. The irradiation time control section 112 may perform a control process so that the irradiation time of the special light is longer than the irradiation time of the white light. The light detection section 107 may detect first return light from the observation target when the white light has been applied to the observation target, and may detect second return light from the observation target when the special light has been applied to the observation target. The irradiation section may acquire the white light using the first filter, and may acquire the special light using the second filter.

The above configuration makes it possible to set the irradiation time of the special light to be longer than the irradiation time of the white light when applying the white light and the special light in a spot-like shape. Therefore, since the irradiation quantity (intensity per unit time×irradiation time) of the special light can be increased as compared with the normal light, it is possible to implement a scanning optical device (e.g., scanning endoscope in a narrow sense) that can prevent a situation in which the brightness of an image (second image in a broad sense) corresponding to the specific wavelength band is insufficient, and can generate a clear image. Since the white light and the special light are acquired by applying the filter to light emitted from a single light source, the configuration of the light source section can be simplified.

The first and second embodiments according to the invention and the modifications thereof have been described above. Note that the invention is not limited to the first and second embodiments and the modifications thereof. Various modifications and variations may be made without departing from the scope of the invention. A plurality of elements described in connection with the first and second embodiments and the modifications thereof may be appropriately combined. For example, some elements may be omitted from the elements described in connection with the first and second embodiments and the modifications thereof. The elements described in connection with the first and second embodiments and the modifications thereof may be appropriately combined. Specifically, various modifications and applications are possible without materially departing from the novel teachings and advantages of the invention.

Any term (e.g., normal light image and special light image) cited with a different term (e.g., first image and second image) having a broader meaning or the same meaning at least once in the specification and the drawings may be replaced by the different term in any place in the specification and the drawings.

What is claimed is:

1. An optical control device that is provided in a scanning optical device that applies light emitted from a light source to an observation target as spot light that is applied in a spot-like shape, and detects return light from the observation target while scanning the spot light,
the optical control device comprising:
an irradiation section that applies white light and special light to the observation target, the special light being light within a specific wavelength band;
an irradiation time control section that performs a control process so that an irradiation time of the special light is longer than an irradiation time of the white light;
a light detection section that detects first return light from the observation target when the white light for which the irradiation time is controlled has been applied to the observation target, and detects second return light from the observation target when the special light for which the irradiation time is controlled has been applied to the observation target; and
an emission control section,
the irradiation section acquiring the white light from a normal light source that emits the white light, acquiring the special light from a special light source that emits the special light, and scanning a scan target area including the observation target using the white light acquired from the normal light source and the special light acquired from the special light source,
the emission control section controlling an emission timing of the normal light source and the special light source so that the irradiation time of the special light is longer than the irradiation time of the white light, and
the light detection section detecting the first return light and the second return light from the observation target when the irradiation section scans the scan target area.

2. The optical control device as defined in claim 1,
the normal light source including first to Nth (N is an integer equal to or larger than 2) monochromatic light sources that respectively emit first to Nth monochromatic lights that form the white light,
the emission control section causing the first to Nth monochromatic light sources to sequentially emit light, and
the irradiation section sequentially acquiring and applying the first to Nth monochromatic lights that form the white light.

3. The optical control device as defined in claim 2,
the first to Nth monochromatic lights being R color light, G color light, and B color light.

4. The optical control device as defined in claim 1,
the special light source including (N+1)th to Mth (M is an integer that satisfies “M>N+1”, and N is an integer) monochromatic light sources that respectively emit (N+1)th to Mth monochromatic lights that form the special light,
the emission control section causing the (N+1)th to Mth monochromatic light sources to sequentially emit light, and
the irradiation section sequentially acquiring and applying the (N+1)th to Mth monochromatic lights that form the special light.

5. The optical control device as defined in claim 1,
the irradiation section scanning entirety of the scan target area using one of the white light and the special light,
the emission control section causing a light source that emits another of the white light and the special light to emit light on condition that the entirety of the scan target area has been scanned using the one of the white light and the special light, and
the irradiation section scanning the entirety of the scan target area using the other of the white light and the special light.

6. The optical control device as defined in claim 5,
the normal light source including first to Nth (N is an integer equal to or larger than 2) monochromatic light sources that respectively emit first to Nth monochromatic lights that form the white light,
the irradiation section scanning the entirety of the scan target area using light emitted from an ith (1≦i≦N−1) monochromatic light source among the first to Nth monochromatic light sources,
the emission control section causing an (i+1)th monochromatic light source among the first to Nth monochromatic light sources to emit light on condition that the entirety of the scan target area has been scanned using the light emitted from the ith monochromatic light source, and
the irradiation section scanning the entirety of the scan target area using light emitted from the (i+1)th monochromatic light source.

7. The optical control device as defined in claim 5,
the special light source including (N+1)th to Mth (M is an integer that satisfies “M>N+1”, and N is an integer) monochromatic light sources that respectively emit (N+1)th to Mth monochromatic lights that form the special light,
the irradiation section scanning the entirety of the scan target area using light emitted from a jth (1≦j≦M−1) monochromatic light source among the (N+1)th to Mth monochromatic light sources,
the emission control section causing a (j+1)th monochromatic light source among the (N+1)th to Mth monochromatic light sources to emit light on condition that the entirety of the scan target area has been scanned using the light emitted from the jth monochromatic light source, and
the irradiation section scanning the entirety of the scan target area using light emitted from the (j+1)th monochromatic light source.

8. The optical control device as defined in claim 1,
the irradiation section applying one of the white light and the special light to an irradiation spot,
the emission control section causing a light source that emits another of the white light and the special light to emit light on condition that the one of the white light and the special light has been applied to the irradiation spot, and
the irradiation section applying the other of the white light and the special light to a next irradiation spot.

9. The optical control device as defined in claim 1,
the emission control section including a cycle control section that controls the emission timing of the normal light source and the special light source so that the normal light source and the special light source alternately emit light in each cycle.

10. The optical control device as defined in claim 1,
the light source being a single light source,
the irradiation section acquiring the white light by applying a first filter that allows the white light to pass through to light emitted from the single light source, and acquiring the special light by applying a second filter that allows the special light to pass through to the light emitted from the single light source, and
the irradiation time control section performing a control process so that an application time of the second filter is longer than an application time of the first filter.

11. The optical control device as defined in claim 10,
the irradiation section sequentially acquiring the white light and the special light by rotating a rotary filter that includes the first filter and the second filter, and
the rotary filter being configured so that the second filter has a size larger than that of the first filter.

12. The optical control device as defined in claim 1,
the specific wavelength band being narrower than a wavelength band of the white light.

13. The optical control device as defined in claim 1,
the specific wavelength band being a wavelength band of light absorbed by hemoglobin in blood.

14. The optical control device as defined in claim 13,
the specific wavelength band being 390 to 445 nm or 530 to 550 nm.

15. The optical control device as defined in claim 1,
the specific wavelength band being a wavelength band of excitation light that causes a fluorescent substance to produce fluorescence.

16. The optical control device as defined in claim 15,
the specific wavelength band being a wavelength band of excitation light that causes the fluorescent substance to produce fluorescence within a wavelength band of 490 to 625 nm, the wavelength band of the excitation light being 390 to 470 nm.

17. The optical control device as defined in claim 1,
the specific wavelength band being a wavelength band of infrared light.

18. The optical control device as defined in claim 17,
the specific wavelength band being 790 to 820 nm or 905 to 970 nm.

19. The optical control device as defined in claim 1,
the scanning optical device being a scanning endoscope.

20. A control device comprising:
an optical control section that is the optical control device as defined in claim 1; and
an image processing section that generates an output image based on an optical signal acquired by the optical control section,
the image processing section generating the output image using the first return light and the second return light detected by the light detection section.

21. The control device as defined in claim 20,
the image processing section including:
an information acquisition section that acquires light identification information that specifies a type of the light applied to the observation target;
a separation section that separates the return light from the observation target into the first return light and the second return light based on the light identification information; and
an image generation section that generates the output image based on the first return light and the second return light separated by the separation section.

22. The control device as defined in claim 21,
the light detection section detecting the first return light when the irradiation section has applied the white light to the observation target, and detecting the second return light when the irradiation section has applied the special light to the observation target.

23. The control device as defined in claim 22,
the image generation section generating a first image based on the first return light detected by the light detection section, generating a second image based on the second return light detected by the light detection section, and generating the output image from the first image and the second image.

24. The control device as defined in claim 21,
the information acquisition section acquiring the light identification information that specifies whether light applied to an irradiation spot is monochromatic light among first to Nth (N is an integer equal to or larger than 2) monochromatic lights that form the white light or monochromatic light among (N+1)th to Mth (M is an integer that satisfies “M>N+1”) monochromatic lights that form the special light, and
the separation section separating the return light into first to Nth monochromatic return lights that correspond to the first to Nth monochromatic lights that form the white light and (N+1)th to Mth monochromatic return lights that correspond to the (N+1)th to Mth monochromatic lights that form the special light based on the light identification information.

25. The control device as defined in claim 24,
the light detection section detecting the first to Nth monochromatic return lights when the irradiation section has applied the first to Nth monochromatic lights to the observation target, and
the image generation section generating first to Nth monochromatic images that form a first image based on the first to Nth monochromatic return lights detected by the light detection section.

26. The control device as defined in claim 24,
the light detection section detecting the (N+1)th to Mth monochromatic return lights when the irradiation section has applied the (N+1)th to Mth monochromatic lights to the observation target, and
the image generation section generating (N+1)th to Mth monochromatic images that form a second image based on the (N+1)th to Mth monochromatic return lights detected by the light detection section.

27. The control device as defined in claim 21,
the irradiation section spirally applying the spot light to the observation target,
the information acquisition section acquiring position information about the spot light,
the image generation section including a first interpolation section and a second interpolation section,
the first interpolation section converting a format of a first image signal that corresponds to the first return light separated by the separation section into a raster scan format based on the position information about the spot light,
the second interpolation section converting a format of a second image signal that corresponds to the second return light separated by the separation section into a raster scan format based on the position information about the spot light, and
the image generation section generating a first image based on the first image signal that has been converted into the raster scan format, and generating a second image based on the second image signal that has been converted into the raster scan format.

28. An optical scope that allows the white light applied by the irradiation section included in the optical control device as defined in claim 1 to pass through, and transmits the return light from the observation target to the light detection section.
